SEQUENCE LISTING (43 sequences)

SEQ ID NO: 1 - protein, 41 aa, synthetic construct
  REGION 1..41: note = FC-epsilon-RI-gamma cytoplasmic domain
LKIQVRKAAITSYEKSDGVYTGLSTRNQETYETLKHEKPPQ

SEQ ID NO: 2 - DNA, 123 bp, synthetic construct
  misc_feature 1..123: note = FC-epsilon-RI-gamma cytoplasmic domain
  source 1..123: mol_type = other DNA
ctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 3 - protein, 54 aa, synthetic construct
  REGION 1..54: note = CD8 hinge
KPTTTPAPRPPTPAPTIASQPLSLRPEACRPAAGGAVHTRGLDFACFWVLVVVG

SEQ ID NO: 4 - protein, 20 aa, synthetic construct
  REGION 1..20: note = CD28 transmembrane domain
GVLACYSLLVTVAFIIFWVR

SEQ ID NO: 5 - protein, 41 aa, synthetic construct
  REGION 1..41: note = CD28 signaling domain
RSKRSRLLHSDYMNMTPRRPGPTRKHYQPYAPPRDFAAYRS

SEQ ID NO: 6 - protein, 42 aa, synthetic construct
  REGION 1..42: note = 4-1BB signaling domain
KRGRKKLLYIFKQPFMRPVQTTQEEDGCSCRFPEEEEGGCEL

SEQ ID NO: 7 - protein, 113 aa, synthetic construct
  REGION 1..113: note = CD3 zeta signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - DNA, 339 bp, synthetic construct
  misc_feature 1..339: note = CD3 zeta
  source 1..339: mol_type = other DNA
gtgaagtttagcagatctgccgacgcccctgcctaccagcagggacagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 9 - protein, 153 aa, synthetic construct
  REGION 1..153: note = Human IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 160 aa, synthetic construct
  REGION 1..160: note = Human er-IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 11 - protein, 254 aa, synthetic construct
  REGION 1..254: note = FCgamma/CD16
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 12 - DNA, 765 bp, synthetic construct
  misc_feature 1..765: note = FCgamma/CD16 nt
  source 1..765: mol_type = other DNA
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 13 - DNA, 1125 bp, synthetic construct
  misc_feature 1..1125: note = CCR7
  source 1..1125: mol_type = other DNA
gccaccatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccatag

SEQ ID NO: 14 - DNA, 297 bp, synthetic construct
  misc_feature 1..297: note = CCL19
  source 1..297: mol_type = other DNA
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaa

SEQ ID NO: 15 - DNA, 405 bp, synthetic construct
  misc_feature 1..405: note = CCL21
  source 1..405: mol_type = other DNA
atggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatag

SEQ ID NO: 16 - DNA, 1083 bp, synthetic construct
  misc_feature 1..1083: note = CXCR2 nt
  source 1..1083: mol_type = other DNA
atggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaa

SEQ ID NO: 17 - protein, 360 aa, synthetic construct
  REGION 1..360: note = CXCR2 aa
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 18 - DNA, 336 bp, synthetic construct
  misc_feature 1..336: note = CXCL4 nt
  source 1..336: mol_type = other DNA
atgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 19 - protein, 111 aa, synthetic construct
  REGION 1..111: note = CXCL4 aa
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 20 - DNA, 1119 bp, synthetic construct
  misc_feature 1..1119: note = CD62L nt
  source 1..1119: mol_type = other DNA
atgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaa

SEQ ID NO: 21 - protein, 372 aa, synthetic construct [sequence omitted]

SEQ ID NO: 22 - DNA, 300 bp, synthetic construct
  misc_feature 1..300: note = IL8 nt
  source 1..300: mol_type = other DNA
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataa

SEQ ID NO: 23 - protein, 99 aa, synthetic construct
  REGION 1..99: note = IL8 aa
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 24 - DNA, 324 bp, synthetic construct
  misc_feature 1..324: note = CXCL1 nt
  source 1..324: mol_type = other DNA
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactga

SEQ ID NO: 25 - protein, 107 aa, synthetic construct
  REGION 1..107: note = CXCL1 aa
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 26 - DNA, 762 bp, synthetic construct
  misc_feature 1..762: note = IL-12 p35 nt
  source 1..762: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 27 - protein, 253 aa, synthetic construct
  REGION 1..253: note = IL-12 p35 aa
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - DNA, 987 bp, synthetic construct
  misc_feature 1..987: note = IL-12 p40 nt
  source 1..987: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 29 - protein, 328 aa, synthetic construct
  REGION 1..328: note = IL-12 p40 aa
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 30 - DNA, 1233 bp, synthetic construct
  misc_feature 1..1233: note = HER2 CAR (Fce)
  source 1..1233: mol_type = other DNA
atggactggatctggcggattctgtttctcgtgggagctgccacaggcgctcattctgctcagcctgccgacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagtcctcagggggcgggggaagtggtgggggcggcagcggcggagggggctcaggaggaggcggatcaggcggatcagaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagcgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 31 - protein, 536 aa, synthetic construct
  REGION 1..536: note = IL-12 single chain p40_p35 aa
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 32 - DNA, 1611 bp, synthetic construct
  misc_feature 1..1611: note = IL-12 single chain p40_p35 nt
  source 1..1611: mol_type = other DNA
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 33 - DNA, 1215 bp, synthetic construct
  misc_feature 1..1215: note = EGFR CAR (Fce)
  source 1..1215: mol_type = other DNA
atggactggatctggcggattctgtttctcgtgggagctgccacaggcgctcattctgctcagcctgccgatattcttcttactcaatctcccgttattttgtcagtatccccaggtgagcgagtcagcttctcttgtcgagcgtcacaatccattggcaccaacatacattggtaccaacagcgcaccaacgggtctccccggctcttgattaagtacgcatcagaaagtatttctgggatacccagtaggttctcagggagcgggagtggcactgactttaccctgtccataaacagcgttgagtctgaggacatcgcggactactattgtcagcagaacaacaattggccgaccacgtttggtgcgggaacaaaacttgaactcaaaggcggcggaggaagcggaggcggaggatctgggggcggaggctctggcggagggggatctcaggtgcagctcaaacagtcaggacctggcctcgttcagccaagccaatcactgagtataacgtgcacggtgagcggctttagcctgacaaactatggtgtccactgggtccgccaatctcctggaaaaggcttggagtggctcggtgttatctggtccggtggtaacacagactacaacacgccattcaccagtcgccttagtattaacaaggacaactccaagtctcaggttttctttaaaatgaactctctgcagtctaatgataccgcaatttactactgtgcgagggcactcacgtactatgactatgagttcgcgtattggggccaagggactctcgttactgtctcagcggcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 34 - DNA, 477 bp, synthetic construct
  misc_feature 1..477: note = TGF-beta receptor II extracellular domain nt
  source 1..477: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 35 - protein, 159 aa, synthetic construct
  REGION 1..159: note = TGF-beta receptor II extracellular domain aa
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 36 - DNA, 933 bp, synthetic construct
  misc_feature 1..933: note = TGF-beta trap nt
  source 1..933: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggaggaagtggaggaggtggaagtggtggtggaggtagtacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 37 - protein, 311 aa, synthetic construct
  REGION 1..311: note = TGF-beta trap aa
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 38 - DNA, 510 bp, synthetic construct
  misc_feature 1..510: note = erIL-15 nt
  source 1..510: mol_type = other DNA
atgcgcatcagcaagccccacctgcgcagcatcagcatccagtgctacctgtgcctgctgctgaacagccacttcctgaccgaggccggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggctccgagaaggacgagctgtaa

SEQ ID NO: 39 - protein, 169 aa, synthetic construct
  REGION 1..169: note = erIL-15 aa
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSEKDEL

SEQ ID NO: 40 - DNA, 807 bp, synthetic construct
  misc_feature 1..807: note = Codon-optimized EGFR scFv - DNA sequence
  source 1..807: mol_type = other DNA
atggactggatctggcggattctgtttctcgtgggagctgccacaggcgctcattctgctcagcctgccgatattcttcttactcaatctcccgttattttgtcagtatccccaggtgagcgagtcagcttctcttgtcgagcgtcacaatccattggcaccaacatacattggtaccaacagcgcaccaacgggtctccccggctcttgattaagtacgcatcagaaagtatttctgggatacccagtaggttctcagggagcgggagtggcactgactttaccctgtccataaacagcgttgagtctgaggacatcgcggactactattgtcagcagaacaacaattggccgaccacgtttggtgcgggaacaaaacttgaactcaaaggcggcggaggaagcggaggcggaggatctgggggcggaggctctggcggagggggatctcaggtgcagctcaaacagtcaggacctggcctcgttcagccaagccaatcactgagtataacgtgcacggtgagcggctttagcctgacaaactatggtgtccactgggtccgccaatctcctggaaaaggcttggagtggctcggtgttatctggtccggtggtaacacagactacaacacgccattcaccagtcgccttagtattaacaaggacaactccaagtctcaggttttctttaaaatgaactctctgcagtctaatgataccgcaatttactactgtgcgagggcactcacgtactatgactatgagttcgcgtattggggccaagggactctcgttactgtctcagcg

SEQ ID NO: 41 - protein, 269 aa, synthetic construct
  REGION 1..269: note = EGFR scFv - Protein sequence
MDWIWRILFLVGAATGAHSAQPADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 42 - DNA, 825 bp, synthetic construct
  misc_feature 1..825: note = Codon-optimized HER2/neu scFv - DNA sequence
  source 1..825: mol_type = other DNA
atggactggatctggcggattctgtttctcgtgggagctgccacaggcgctcattctgctcagcctgccgacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagtcctcagggggcgggggaagtggtgggggcggcagcggcggagggggctcaggaggaggcggatcaggcggatcagaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagc

SEQ ID NO: 43 - protein, 275 aa, synthetic construct
  REGION 1..275: note = HER2/neu scFv - Protein sequence
MDWIWRILFLVGAATGAHSAQPADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSGGGGSGGGGSGGGGSGGGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

(19) United States
(12) Patent Application Publication
(10) Pub. No.: US 2025/0154465 A1
(43) Pub. Date: May 15, 2025

(54) CHIMERIC ANTIGEN RECEPTOR-MODIFIED NK-92 CELLS TARGETING EGFR SUPER-FAMILY RECEPTORS

(71) Applicant: ImmunityBio, Inc., San Diego, CA (US)

(72) Inventors: John H. Lee, Culver City, CA (US); Laurent H. Boissel, Culver City, CA (US); Hans G. Klingemann, Culver City, CA (US)

(73) Assignee: ImmunityBio, Inc., San Diego, CA (US)

(21) Appl. No.: 19/034,027

(22) Filed: Jan. 22, 2025

Related U.S. Application Data
(63) Continuation of application No. 17/489,607, filed on Sep. 29, 2021.
Application No. 17/489,607 is a division of application No. 16/775,111, filed on Jan. 28, 2020, now Pat. No. 11,230,699.

(51) Int. Cl. C12N 5/0783; A61K 40/15; A61K 40/31; A61K 40/42; A61K 45/06; A61P 35/00; C07K 14/54; C07K 14/55; C07K 14/71; C07K 14/735; C07K 16/28; C07K 16/32

(52) U.S. Cl. CPC C12N 5/0646; A61K 40/15; A61K 40/31; A61K 40/4204; A61K 40/4205; A61K 45/06; A61P 35/00; C07K 14/5434; C07K 14/5443; C07K 14/55; C07K 14/70535; C07K 14/71; C07K 16/2863; C07K 16/32; A61K 2239/31; A61K 2239/38; A61K 2239/50; C07K 2317/24; C07K 2317/53; C07K 2317/622; C07K 2317/732; C07K 2319/02; C07K 2319/03; C07K 2319/30; C07K 2319/33

(57) ABSTRACT

Provided are genetically modified NK cells expressing a chimeric antigen receptor targeting an EGFR superfamily receptor. The CAR can comprise an intracellular domain of FcεRIγ and further recombinant proteins expressed by the genetically modified NK cells are CD16, autocrine growth stimulating cytokines, and optionally one of IL-12, a TGF-beta trap, or a homing receptor. Also described are methods for treating a patient having or suspected of having a disease that is treatable with NK-92 cells, such as cancer, comprising administering to the patient the genetically modified NK cells.

### RELATED APPLICATIONS

This application is a continuation application of allowed U.S. patent application with the Ser. No. 17/489,607, filed Sep. 29, 2021, which claims priority to U.S. Pat. No. 11,230,699 having Ser. No. 16/775,111, filed Jan. 28, 2020, both of which are incorporated by reference in their entirety.

### SEQUENCE LISTING

The content of the XML file of the sequence listing named 104077.0007US8_Sequence List, which is 72.4 kb in size was created on Jan. 22, 2025 and electronically submitted via Patent Center along with the application is incorporated by reference in its entirety.

### FIELD OF THE INVENTION

The field of the invention is genetically modified immune competent cells that express a chimeric antigen receptor (CAR), and particularly modified NK-92 cells expressing a CAR with an Fc epsilon receptor gamma (FcεRIγ) signaling domain targeting an EGFR superfamily receptor.

### BACKGROUND OF THE INVENTION

Natural killer (NK) cells are cytotoxic lymphocytes that constitute a significant component of the innate immune system. In most cases, NK cells represent about 10-15% of circulating lymphocytes, and bind and kill targeted cells, including virus-infected cells and many malignant cells. NK cell killing is non-specific with regard to particular antigens and can occur without prior immune sensitization. Killing of targeted cells is typically mediated by cytolytic proteins, including perforin, granzymes, and granulysin.

Autologous NK cells have been used as therapeutic entities. To that end, NK cells are isolated from the peripheral blood lymphocyte fraction of whole blood, expanded in cell culture to obtain sufficient numbers of cells, and then re-infused into a subject. Autologous NK cells have shown in at least some cases moderate effectiveness in both ex vivo therapy and in vivo treatment. However, isolation and growth of autologous NK cell is time and cost intensive. Moreover, autologous NK cell therapy is further limited by the fact that not all NK cells are cytolytic.

At least some of these difficulties can be overcome by use of NK-92 cells, which are a cytolytic cancer cell line which was discovered in the blood of a subject suffering from a non-Hodgkins lymphoma and then immortalized in vitro (Gong et al., Leukemia 8:652-658 (1994)). While NK-92 cells are NK cell derivatives, NK-92 cells lack the major of inhibitory receptors that are otherwise displayed by normal NK cells, and retain the majority of the activating receptors. NK-92 cells do not, however, attack normal cells nor do they elicit an unacceptable immune rejection response in humans. Due to these desirable characteristics, NK-92 cells were characterized in detail and explored as therapeutic agent in the treatment of certain cancers as is described, for example, in WO 1998/049268 or US 2002/068044.

Phenotypic changes distinguishing a tumor cell from normal cells derived from the same tissue are often associated with one or more changes in the expression of specific gene products, including the loss of normal cell surface components or the gain of others (i.e., antigens not detectable in corresponding normal, non-cancerous tissue). The antigens which are expressed in neoplastic or tumor cells, but not in normal cells, or which are expressed in neoplastic cells at levels substantially above those found in normal cells, have been termed “tumor-specific antigens” or “tumor-associated antigens.” Such tumor-specific antigens may serve as markers for tumor phenotype. Tumor-specific antigens include cancer/testis-specific antigen (e.g. MAGE, BAGE, GAGE, PRAME and NY-ESO-1), melanocyte differentiation antigens (e.g. tyrosinase, Melan-A/MART, gp100, TRP-1 and TRP-2), mutated or aberrantly expressed antigens (e.g. MUM-1, CDK4, beta-catenin, gp100-in4, p15 and N-acetylglucos-aminyltransferase V), and antigens that are expressed at higher levels in tumors (e.g., CD19 and CD20).

Tumor-specific antigens have been used as targets for cancer immunotherapies. One such therapy utilizes chimeric antigen receptors (CARs) expressed on the surface of immune cells, including T cells and NK cells, to improve cytotoxicity against cancer cells. CARs comprise a single-chain variable fragment (scFv) linked to at least one intracellular signaling domain. The scFv recognizes and binds an antigen on the target cell (e.g., a cancer cell) and triggers effector cell activation. The signaling domains contain immunoreceptor tyrosine-based activation domains (ITAMs) that are important for intracellular signaling by the receptor.

The first generation of CARs used in T-cells contained one cytoplasmic signaling domain. For example, one version of a first-generation CAR in T-cells included a signaling domain from the Fc epsilon receptor gamma (FcεRIγ) which contained one ITAM, while another version contained the signaling domain from CD3ζ which contained three ITAMs. In vivo and in vitro studies showed that the CD3ζ CAR T-cells were more efficient at tumor eradication than FcεRIγ CAR T-cells (e.g., Haynes, et al. 2001, J. Immunology 166:182-187; Cartellieri, et al. 2010, J. Biomed and Biotech, Vol. 2010, Article ID 956304). Additional studies then revealed that certain costimulatory signals were required for full activation and proliferation of such recombinant T-cells, and second and third generation CARs combined multiple signaling domains in to a single CAR to enhance efficacy of the recombinant CAR T-cells. Due to their less desirable philological effects in the tested T-cells, first generation CARs and the FcεRIγ signaling domains were largely discarded in favor of the new, more efficient CARs using CD3ζ in combination with one or more additional signaling domains (e.g., Hermanson and Kaufman 2015, Frontiers in Immunol., Vol. 6, Article 195).

More recently, selected CARs have also been expressed in NK cells. For example, CAR-modified NK-92 cells have used first generation CARs with only a CD3ζ intracellular signaling domain. Several antigens have been targeted by these first generation CAR-NK cells, including CD19 and CD20 for B cell lymphoma, ErbB2 for breast, ovarian, and squamous cell carcinoma, GD2 for neuroblastoma, and CD138 for multiple myeloma. Second generation CAR-NK cells from the NK-92 line have also been created for several antigens, including EpCAM for multiple carcinomas HLA-A2 EBNA3 complex for Epstein-Barr virus, CS1 for multiple myeloma, and ErbB2 for HER2 positive epithelial cancers. The most common intracellular costimulatory domain used alongside CD3ζ in second generation NK-92 CARs is CD28. However, the potential effect of the CD28 domain is unclear since NK cells do not naturally express CD28. Additional second generation CARs have incorporated the 4-1BB intracellular signaling domain along with CD3ζ to improve NK cell persistence. Others compared functionality of different intracellular domains using an ErbB2 scFv fused with CD3ζ alone, CD28 and CD3ζ, or 4-1BB and CD3ζ tested against breast cancer cells. They found that both of the second generation constructs improved killing compared to the first generation CARs and the CD28 and CD3ζ had 65% target lysis, the 4-1BB and CD3ζ lysed 62%, and CD3ζ alone killed 51% of targets. 4-1BB and CD28 intracellular domains were also compared in a recent study using anti-CD19 CARs expressed on NK-92 cells for B cell malignances. Still others found that CD3ζ/4-1BB constructs were less effective than CD3ζ/CD28 in cell killing and cytokine production, highlighting differential effects of CD28 and 4-1BB costimulatory domains.

Third generation NK-92 CARs were constructed of an anti-CD5 scFv with CD3ζ, CD28, and 4-1BB intracellular signaling domains and demonstrated specific and potent anti-tumor activity against a variety of T-cell leukemia and lymphoma cell lines and primary tumor cells. Such cells were also able to inhibit disease progression in xenograft mouse models of T cell Acute lymphoblastic leukemia (ALL) cell lines as well as primary tumor cells (Transl Res. 2017 September; 187: 32-43). In further examples, WO 2016/201304 and WO 2018/076391 teach use of third generation CD3ζ CARs expressed in NK cells and NK-92 cells.

However, NK cells (and particularly NK-92 cells) are often difficult to genetically modify as evidenced by numerous failures to engineer NK-92 cells to express an Fc receptor. Such difficulties are further compounded where NK-92 cells are transfected with multiple recombinant genes or relatively large recombinant nucleic acid payload for heterologous expression. Additionally, NK-92 cells also exhibit a significant lack of predictability with respect to recombinant expression of exogenous proteins (e.g., CD16). On a functional level, while exhibiting in most cases targeted cytotoxicity, most if not all CAR NK-92 cells require a high effector to target cell ratio.

In addition, even where cytotoxic cells expressing a CAR are relatively effective in vitro, various suppressive or inhibitory factors associated with the tumor microenvironment in vivo may reduce or even abrogate cytotoxicity of such cells. Similarly, and despite the expression of a CAR, such modified cells may not always be effective in targeting the tumor microenvironment.

Therefore, even though numerous recombinant NK-92 cells are known in the art, all or almost all of them suffer from various difficulties. Consequently, there remains a need for CAR-expressing NK-92 cells that express a high-activity CAR in significant quantities, that can be readily cultivated in a simple and effective manner, and that have high cytotoxicity in a tumor microenvironment.

### SUMMARY OF THE INVENTION

The inventors have discovered that natural killer (NK) cells, and particularly NK-92 cells, may be genetically modified to express a target-specific CAR and further recombinant proteins to increase CAR mediated cell killing, ADCC, and cytotoxicity in and/or homing to a tumor microenvironment. In addition, such recombinant cells also expressed cytokines for autocrine growth stimulation that advantageously assists in clonal selection of modified cells.

In one aspect of the inventive subject matter, the inventors contemplate a genetically modified NK cell (and especially an NK-92 cell) that expresses (i) a membrane bound recombinant chimeric antigen receptor (CAR) that comprises in a single polypeptide chain an extracellular binding domain, a hinge domain, a transmembrane domain, and a signaling domain, wherein the extracellular binding domain specifically binds to an EGFR superfamily receptor; (ii) a recombinant CD16; (iii) an autocrine growth stimulating cytokine; and (iv) optionally one of IL-12, a TGF-beta trap, or a homing receptor.

In some embodiments, the extracellular binding domain comprises a scFv, and/or the signaling domain comprises a FcεRIγ signaling domain. Most typically, the EGFR superfamily receptor is HER-2 or EGFR. In further contemplated aspects, the recombinant CD16 is a CD16158V mutant, and/or the autocrine growth stimulating cytokine is IL-2 or IL-15, which may additionally comprise an endoplasmic retention sequence. In still further contemplated embodiments, the IL-12 is a single chain IL-12 heterodimer, and the TGF-beta trap comprises a single chain dimer of the TGF-beta Receptor II ectodomain and is preferably secreted. Preferred homing receptors include cell adhesion molecules, selectins, integrins, a C-C chemokine receptor, or a C-X-C chemokine receptor. For example, suitable homing receptor include CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCXCKR, D6, DARC, and the receptor for CXCL14.

Viewed from a different perspective, genetically modified NK cells are contemplated that include a nucleic acid that encodes a (i) membrane bound recombinant chimeric antigen receptor (CAR) that comprises in a single polypeptide chain an extracellular binding domain, a hinge domain, a transmembrane domain, and a signaling domain, wherein the extracellular binding domain specifically binds to a EGFR superfamily receptor; (ii) a recombinant CD16; (iii) an autocrine growth stimulating cytokine; and (iv) optionally one of IL-12, a TGF-beta trap, or a homing receptor. With regard to the NK cell and the expressed proteins, the same considerations as noted above apply.

In another aspect of the inventive subject matter, the inventors also contemplate a method of treating cancer in a patient in need thereof in which the patient receives a therapeutically effective amount of the genetically modified NK cells presented herein, thereby treating the cancer. Where desired, contemplated methods may also include a step of administering at least one additional therapeutic entity selected from the group consisting of a viral cancer vaccine, a bacterial cancer vaccine, a yeast cancer vaccine, N-803, an antibody, a stem cell transplant, and a tumor targeted cytokine. For example, contemplated cancers include lung cancer, a breast cancer, a thyroid cancer, an esophageal cancer, a gastric cancer, a gastroesophageal cancer, or a head and neck cancer. Most typically, about 1×10⁸ to about 1×10¹¹ cells per m² of body surface area of the patient are administered to the patient. Therefore, the inventors also contemplate use of a genetically modified NK cell in the treatment of cancer.

Various objects, features, aspects and advantages of the inventive subject matter will become more apparent from the following detailed description of preferred embodiments along with the accompanying drawing figures in which like numerals represent like components.

### BRIEF DESCRIPTION OF THE DRAWINGS

FIG. 1 is a schematic representation of exemplary CAR constructs. All CAR variants had an extracellular domain comprising an anti-EGFR superfamily receptor scFv region (e.g., αEGFR-scFv, αHER2-scFv), a hinge region from CD8 (CD8 hinge), and a transmembrane domain from CD28 (CD28 TM). The intracellular domains of the CD19CARs were varied as indicated.

FIG. 2 is a schematic representation of an exemplary tricistronic recombinant nucleic acid encoding a HER2.CAR, followed by a P2A sequence, followed by a sequence encoding a high-affinity variant of CD16. The CD16 sequence is followed by an IRES sequence, which is followed in turn by a sequence encoding erIL-2.

FIG. 3 is another schematic representation of an exemplary tricistronic recombinant nucleic acid encoding a HER2.CAR, a high-affinity variant of CD16, and erIL-2 used to generate recombinant NK cells.

FIG. 4 depicts exemplary FACS results for NK cells transfected with the tricistronic recombinant nucleic acid demonstrating expression of CD16 and HER2.CAR from a polyclonal collection of cells.

FIG. 5 depicts exemplary results for CAR-mediated cytotoxicity of NK cells transfected with the tricistronic recombinant nucleic acid of FIG. 3.

FIG. 6 depicts exemplary results for ADCC of NK cells transfected with the tricistronic recombinant nucleic acid of FIG. 3.

FIG. 7 depicts exemplary FACS results for NK cells transfected with the tricistronic recombinant nucleic acid demonstrating expression of CD16 and HER2.CAR from selected clonal cell lines.

FIG. 8 depicts exemplary results for natural cytotoxicity of NK cells from selected clonal cell lines transfected with the tricistronic recombinant nucleic acid of FIG. 3.

FIG. 9 depicts exemplary results for CAR-mediated cytotoxicity of NK cells from selected clonal cell lines transfected with the tricistronic recombinant nucleic acid of FIG. 3.

FIG. 10 depicts further exemplary results for CAR-mediated cytotoxicity of NK cells from selected clonal cell lines transfected with the tricistronic recombinant nucleic acid of FIG. 3.

FIG. 11 depicts further exemplary results for ADCC of NK cells from selected clonal cell lines transfected with the tricistronic recombinant nucleic acid of FIG. 3.

FIG. 12 depicts exemplary results for erIL-2 release from NK cells from selected clonal cell lines transfected with the tricistronic recombinant nucleic acid of FIG. 3.

FIG. 13 depicts exemplary in vivo results for MDA-MB-453 tumor volume changes using NK cells transfected with the tricistronic recombinant nucleic acid of FIG. 3.

FIG. 14 depicts exemplary in vivo results for BT-474 tumor volume changes using NK cells transfected with the tricistronic recombinant nucleic acid of FIG. 3.

FIG. 15 is a schematic representation of an exemplary tricistronic recombinant nucleic acid encoding a EGFR2.CAR, followed by a P2A sequence, followed by a sequence encoding a high-affinity variant of CD16. The CD16 sequence is followed by an IRES sequence, which is followed in turn by a sequence encoding erIL-2.

FIG. 16 is another schematic representation of an exemplary tricistronic recombinant nucleic acid encoding a EGFR2.CAR, a high-affinity variant of CD16, and erIL-2 used to generate recombinant NK cells.

FIG. 17 depicts exemplary FACS results for NK cells transfected with the tricistronic recombinant nucleic acid demonstrating expression of CD16 and EGFR2.CAR from a polyclonal collection of cells.

FIG. 18 depicts exemplary results from a polyclonal collection of cells for natural cytotoxicity of NK cells transfected with the tricistronic recombinant nucleic acid of FIG. 16.

FIG. 19 depicts exemplary results from a polyclonal collection of cells for CAR-mediated cytotoxicity of NK cells transfected with the tricistronic recombinant nucleic acid of FIG. 16.

FIG. 20 depicts exemplary results from a polyclonal collection of cells for ADCC of NK cells transfected with the tricistronic recombinant nucleic acid of FIG. 16.

FIG. 21 depicts exemplary results from selected cell lines for natural cytotoxicity of NK cells transfected with the tricistronic recombinant nucleic acid of FIG. 16.

FIG. 22 depicts exemplary results from selected cell lines for CAR-mediated cytotoxicity of NK cells transfected with the tricistronic recombinant nucleic acid of FIG. 16.

FIG. 23 depicts further exemplary results from selected cell lines for CAR-mediated cytotoxicity of NK cells transfected with the tricistronic recombinant nucleic acid of FIG. 16

FIG. 24 depicts further exemplary results for ADCC of NK cells from selected clonal cell lines transfected with the tricistronic recombinant nucleic acid of FIG. 16.

FIG. 25 depicts exemplary results for erIL-2 release from NK cells from selected clonal cell lines transfected with the tricistronic recombinant nucleic acid of FIG. 16.

FIG. 26 is a schematic representation of an exemplary quadracistronic recombinant nucleic acid encoding a TGF-beta trap, a CAR, a high-affinity variant of CD16, and erIL-2 used to generate recombinant NK cells.

FIG. 27 depicts exemplary results for expression of the TGF-beta trap in NK cells transfected with the quadracistronic recombinant nucleic acid of FIG. 26.

FIG. 28 is a schematic representation of an exemplary quadracistronic recombinant nucleic acid encoding an IL-12 heterodimer, a CAR, a high-affinity variant of CD16, and erIL-2 used to generate recombinant NK cells.

FIG. 29 is a schematic representation of IL-12 heterodimers and respective expression levels of the IL-12 heterodimers in NK cells transfected with the quadracistronic recombinant nucleic acid of FIG. 28.

FIG. 30 is a schematic representation of an exemplary quadracistronic recombinant nucleic acid encoding CCR7, a CAR, a high-affinity variant of CD16, and erIL-2 used to generate recombinant NK cells.

### DETAILED DESCRIPTION OF THE INVENTION

The inventors have discovered that genetically modified NK cells can have significant general toxicity, target specific CAR-mediated cytotoxicity, and ADCC (antibody dependent cellular cytotoxicity), and that such genetically modified cells can be grown under autocrine growth stimulation that will also confer selective effect towards successfully transfected cells. In addition, contemplated cells may further express from a recombinant nucleic acid secreted IL-12 and/or a TGF-beta trap to reduce or eliminate an immune-suppressive environment. Additionally, or alternatively, contemplated cells may express from a recombinant nucleic acid a homing receptor. Therefore, modified cells may be generated by transfection with a tricistronic or quadracistronic nucleic acid.

It should be further appreciated that FcεRIγ-containing CARs have not been utilized in NK-92 cells, other NK cell lines, or endogenous NK cells as other signaling domains (e.g., CD3ζ) were deemed more efficient, especially when combined with additional signaling domains (in second and third generation CARs). Notably, the inventors have discovered that NK-92 cells expressing a first-generation CAR comprising an intracellular domain from FcεRIγ, which has only one ITAM domain, have equal or higher cytotoxic activity against cancer cells expressing the antigen recognized by the CAR than NK-92 cells expressing CARs with a CD3ζ signaling domain, which has three ITAM domains, even where these ITAM domains were combined with other signaling domains (i.e., second or third generation CARs). The inventors also made the unexpected finding that a CAR comprising an intracellular domain from FcεRIγ was expressed at higher levels on the surface of NK-92 cells than other CARs, especially those comprising the CD3ζ signaling domain. Cytotoxic effects can be even further enhanced by expression and secretion of IL-12, which reduces immune suppression in the tumor microenvironment (e.g., via exocrine stimulation of NK cells, modulation of MDSC, etc.), and/or by expression and secretion or presentation of a TGF-beta trap. Alternatively, or additionally, cytotoxic effects can also be enhanced by expression of one or more homing receptors to attract and/or retain NK cells in the tumor microenvironment.

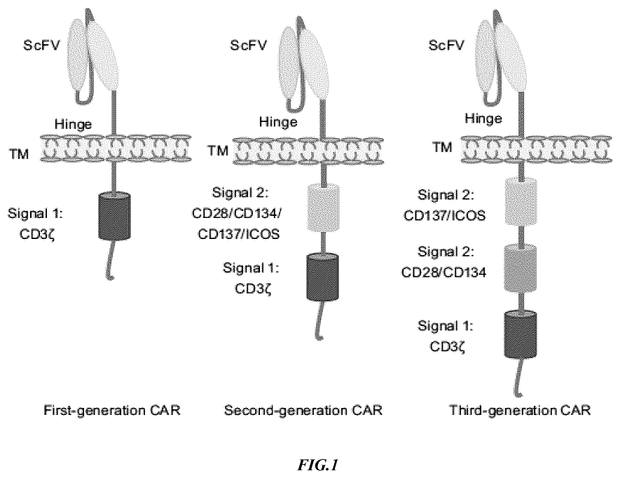

Therefore, in some aspects of the inventive subject matter, a genetically modified NK-92 cell or NK cell line is engineered to express a chimeric antigen receptor (CAR) on a cell surface, and particularly a CAR that specifically binds to an EGFR superfamily receptor such as EGFR or HER2. Most typically, the CAR comprises an intracellular domain from the Fc epsilon receptor gamma (FcεRIγ). However, in further contemplated embodiments the CAR may also comprise a T cell receptor (TCR) CD3 zeta (CD3ζ) intracellular domain, alone or in combination with additional components as are known from 2^{nd} and 3^{rd} generation CAR constructs (e.g., CD28, CD134, CD137, and/or ICOS). As will be readily appreciated, the CAR may be transiently or stably expressed by the NK-92 cell from a recombinant DNA or RNA molecule. Exemplary CAR constructs suitable for use herein are shown in FIG. 1.

Consequently, in one aspect of the inventive subject matter, an NK cell, an NK-92 cell or NK/NK-92 cell line expresses a chimeric antigen receptor (CAR) on the surface of the NK-92 cell that comprises a cytoplasmic domain of FcεRIγ (e.g., having amino acid sequence of SEQ ID NO:1). Alternatively, or additionally, the CAR may also comprise a cytoplasmic domain of CD3 zeta (e.g., having amino acid sequence of SEQ ID NO:7, which may be encoded by a nucleic acid of SEQ ID NO:8 (codon optimized)). In another aspect, an NK or NK-92 cell line is contemplated that is transformed with a nucleic acid encoding a chimeric antigen receptor (CAR). For example, preferred nucleic acids encode a cytoplasmic domain of FcεRIγ (e.g., comprising or consisting of SEQ ID NO:2). As discussed in more detail below, the CAR may target a EGFR superfamily receptor such as EGFR or HER2.

In further contemplated embodiments, the NK, NK-92, or other NK cell is modified to express an autocrine growth stimulating cytokine or variant thereof. For example, suitable cytokines may be transiently or stably expressed by the recombinant cell, and the cytokine may include an endoplasmic retention signal. Most typically (but not necessarily) a retention signal will reduce the amount of secreted cytokine and as such may act as an endocrine growth stimulus without producing systemic effects otherwise encountered by the cytokine expression. Beneficially, the nucleic acid sequence encoding the autocrine growth stimulating cytokine or variant thereof is located on the same recombinant nucleic acid, typically as part of a tri- or quadracistronic configuration. Consequently, recombinant cells transfected with the tri- or quadracistronic nucleic acid can be readily selected and propagated by virtue of their independence from otherwise needed exogenous IL-2.

Additionally, it is generally preferred that the genetically modified NK cells will also express a recombinant CD16 or high-affinity variant thereof (e.g., CD16^{158V}) to impart to the cells target specific ADCC. Advantageously, such co-expression with the CAR is thought to further increase cytotoxicity against a tumor cell. In this context, it should be appreciated that unmodified NK cells typically do not express CD16 and exhibit only cytotoxicity as a part of the innate immune system.

More recently it has been discovered that efficacy of immune therapy may be reduced or even entirely abolished by various factors present in the tumor microenvironment. For example, immune suppressive factors include among other players certain cytokines (e.g., TGF-beta) and various suppressive cells (e.g., MDSC). Consequently, genetically modified NK cells may further express one or more recombinant proteins to counteract the immune suppressive factors. For example, and as is described in more detail below, the genetically modified NK cells may express a TGF-beta trap to reduce TGF-beta mediated effects in the tumor microenvironment and/or may express IL-12 to suppress MDSCs. Additionally, or alternatively, the genetically modified NK cells may also express one or more homing receptors to the tumor microenvironment (or other desired tissue) to so increase the number of therapeutic cells in the tumor microenvironment and thus enhance the therapeutic effect.

In another aspect of the inventive subject matter, the inventors also contemplate a method of treating cancer in a patient in need thereof that includes a step of administering to the patient a therapeutically effective amount of modified NK/NK-92 cells or an NK/NK-92 cell line engineered to express a chimeric antigen receptor (CAR) as described herein. Viewed form a different perspective, the inventors also contemplate a modified NK/NK-92 cell or a NK/NK-92 cell line that expresses a chimeric antigen receptor (CAR), preferably comprising a cytoplasmic domain of FcεRIγ, for use in treating a tumor in a subject. In some embodiments, the use comprises administering to the subject effective amounts of modified cells or the cell line described herein to treat the tumor. In yet other embodiments, an in vitro method for killing tumor cells is contemplated and may include a step of contacting a tumor cell with a modified NK-92 cell or NK-92 cell line described herein. In some embodiments, the modified NK-92 cell or NK-92 cell line expresses a CAR that binds to an antigen on the tumor cell. In some embodiments, the CAR preferably comprises an intracellular domain from the Fc epsilon receptor gamma (FcεRIγ). Alternatively, or additionally, the CAR comprises a T cell receptor (TCR) CD3 zeta (CD3ζ) intracellular domain.

With respect to suitable NK cells, it should be noted that all NK cells are deemed suitable for use herein and therefore include primary NK cells (preserved, expanded, and/or fresh cells), secondary NK cells that have been immortalized, autologous or heterologous NK cells (banked, preserved, fresh, etc.), and modified NK cells as described in more detail below. In some embodiments, it is preferred that the NK cells are NK-92 cells. The NK-92 cell line is a unique cell line that was discovered to proliferate in the presence of interleukin 2 (IL-2) (see e.g., Gong et al., Leukemia 8:652-658 (1994)). NK-92 cells are cancerous NK cells with broad anti-tumor cytotoxicity and predictable yield after expansion in suitable culture media. Advantageously, NK-92 cells have high cytolytic activity against a variety of cancers.

The original NK-92 cell line expressed the CD56^{bright}, CD2, CD7, CD11a, CD45, and CD54 surface markers and did not display the CD1, CD3, CD4, CD5, CD8, CD10, CD14, CD16, CD19, CD20, CD23, and CD34 markers. Growth of such NK-92 cells in culture is dependent upon the presence of interleukin 2 (e.g., rIL-2), with a dose as low as 1 IU/mL being sufficient to maintain proliferation. IL-7 and IL-12 do not support long-term growth, nor have various other cytokines tested, including IL-1α, IL-6, tumor necrosis factor α, interferon α, and interferon γ. Compared to primary NK cells, NK-92 typically have a high cytotoxicity even at relatively low effector:target (E:T) ratios, e.g. 1:1. Representative NK-92 cells are deposited with the American Type Culture Collection (ATCC), designation CRL-2407.

Therefore, suitable NK cells may have one or more modified KIR that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest (see URL www.nantkwest.com) as aNK cells (‘activated natural killer cells). Such cells may then be additionally genetically modified to a CAR as further described in more detail below.

In another aspect of the inventive subject matter, the genetically engineered NK cell may also be an NK-92 derivative that is modified to express the high-affinity Fcγ receptor (CD16). Sequences for high-affinity variants of the Fcγ receptor are well known in the art (see e.g., Blood 2009 113:3716-3725; or SEQ ID NO:11 (with V at amino acid position 158) and SEQ ID NO:12 (with codon for V at amino acid position 158)), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells. Such cells may then be additionally genetically modified to a CAR as further described in more detail below.

Therefore, NK cells suitable for use herein include NK-92 cells (which may be transfected with a tricistronic or quadracistronic construct encoding a CAR, a CD16 or variant thereof, and a cytokine or variant thereof, and optionally one of IL-12, a TGF-beta trap, and a homing receptor), a genetically modified NK cell or NK-92 cell that expresses a CD16 or variant thereof or a cytokine or variant thereof (which may be transfected with a nucleic acid encoding a CAR and a CD16 or variant thereof or a cytokine or variant thereof), and a genetically modified NK cell or NK-92 cell that expresses a CD16 or variant thereof and a cytokine or variant thereof (which may be transfected with a nucleic acid encoding a CAR). As noted before, any of the NK cells contemplated herein may express one of IL-12, a TGF-beta trap, and a homing receptor from the same or a different recombinant nucleic acid.

Genetic modification of the NK cells contemplated herein can be performed in numerous manners, and all known manners are deemed suitable for use hereon. Moreover, it should be recognized that NK cells can be transfected with DNA or RNA, and the particular choice of transfection will at least in part depend on the type of desired recombinant cell and transfection efficiency. For example, where it is desired that NK cells are stably transfected, linearized DNA may be introduced into the cells for integration into the genome. On the other hand, where transient transfection is desired, circular DNA or linear RNA (e.g., mRNA with polyA⁺ tail) may be used.

Similarly, it should be appreciated that the manner of transfection will at least in part depend on the type of nucleic acid employed. Therefore, viral transfection, chemical transfection, mechanical transfection methods are all deemed suitable for use herein. For example, in one embodiment, the vectors described herein are transient expression vectors. Exogenous transgenes introduced using such vectors are not integrated in the nuclear genome of the cell; therefore, in the absence of vector replication, the foreign transgenes will be degraded or diluted over time.

In another embodiment, vectors will preferably allow for stable transfection of cells. In one embodiment, the vector allows incorporation of the transgene(s) into the genome of the cell. Preferably, such vectors have a positive selection marker and suitable positive selection markers include any genes that allow the cell to grow under conditions that would kill a cell not expressing the gene. Non-limiting examples include antibiotic resistance, e.g. geneticin (Neo gene from Tn5). Alternatively, or additionally, the vector is a plasmid vector. In one embodiment, the vector is a viral vector. As would be understood by one of skill in the art, any suitable vector can be used, and suitable vectors are well-known in the art.

In still other embodiments, the cells are transfected with mRNA encoding the protein of interest (e.g., the CAR). Transfection of mRNA results in transient expression of the protein or proteins. In one embodiment, transfection of mRNA into NK-92 cells is performed immediately prior to administration of the cells. In one embodiment, “immediately prior” to administration of the cells refers to between about 15 minutes and about 48 hours prior to administration. Preferably, mRNA transfection is performed about 5 hours to about 24 hours prior to administration. In at least some embodiments as described in more detail below, NK cell transfection with mRNA resulted in unexpectedly consistent and strong expression of the CAR at a high faction of transfected cells. Moreover, such transfected cells also exhibited a high specific cytotoxicity at comparably low effector to target cell ratios.

With respect to contemplated CARs it is noted that the NK/NK-92 cells will be genetically modified to express the CAR as a membrane bound protein exposing a portion of the CAR on the cell surface while maintaining the signaling domain in the intracellular space. Most typically, the CAR will include at least the following elements (in order): an extracellular binding domain, a hinge domain, a transmembrane domain, and a signaling domain (preferably, but not necessarily a FcεRIγ domain).

In preferred embodiments, the cytoplasmic domain of the CAR comprises or consists of a signaling domain of FcεRIγ. For example, the FcεRIγ signaling domain comprises or consists of or consists essentially of the amino acid sequence of SEQ ID NO:1. In some embodiments, the FcεRIγ cytoplasmic domain is the sole signaling domain. However, it should be appreciated that additional elements may also be included, such as other signaling domains (e.g., CD28 signaling domain, CD3ζ signaling domain, 4-1BB signaling domain, etc.). These additional signaling domains may be positioned downstream of the FcεRIγ cytoplasmic domain and/or upstream of the FcεRIγ cytoplasmic domain.

In some embodiments, the FcεRIγ signaling domain comprises or consists of or consists essentially of an amino acid sequence having at least about 85%, 90%, 95%, 96%, 97%, 98%, or 99% sequence homology to the amino acid sequence of SEQ ID NO:1.

As noted above, in some embodiments, the cytoplasmic domain of the CAR comprises a signaling domain of CD3 zeta (CD3ζ). In one embodiment, the cytoplasmic domain of the CAR consists of a signaling domain of CD3 zeta. In one embodiment, the CD3 zeta signaling domain comprises or consists of or consists essentially of the amino acid sequence of SEQ ID NO:7. In some embodiments, the CD3 zeta signaling domain comprises or consists of or consists essentially of an amino acid sequence having at least about 85%, 90%, 95%, 96%, 97%, 98%, or 99% sequence homology to the amino acid sequence of SEQ ID NO:7.

The CAR may comprise any suitable transmembrane domain. In one aspect, the CAR comprises a transmembrane domain of CD28. In one embodiment, the CD28 transmembrane domain comprises or consists of or consists essentially of the amino acid sequence of SEQ ID NO:4. In one embodiment, the CD28 transmembrane domain comprises or consists of or consists essentially of an amino acid sequence having at least about 85%, 90%, 95%, 96%, 97%, 98%, or 99% sequence homology to the amino acid sequence of SEQ ID NO:4. In one embodiment, the transmembrane domain is selected from a CD28 transmembrane domain, 4-1BB transmembrane domain, or FcεRIγ transmembrane domain.

The CAR may comprise any suitable hinge region. In one aspect, the CAR comprises a hinge region of CD8. In one embodiment, the CD8 hinge region comprises or consists of or consists essentially of the amino acid sequence of SEQ ID NO:3. In one embodiment, the CD8 hinge region comprises or consists of or consists essentially of an amino acid sequence having at least about 85%, 90%, 95%, 96%, 97%, 98%, or 99% sequence homology to the amino acid sequence of SEQ ID NO:3.

Most typically, but not necessarily, the extracellular binding domain of the CAR will be a scFv or other natural or synthetic binding portion that specifically binds an antigen of interest such as an EGFR superfamily receptor (and particularly EGFR or HER2). Especially suitable binding portions include small antibody fragments with single, dual, or multiple target specificities, beta barrel domain binders, page display fusion proteins, etc. However, in other embodiments, suitable extracellular binding domains will specifically bind to a tumor-specific antigen, a tumor associated antigen, or a patient- and tumor-specific antigen. For example, contemplated antigens include CD19, CD20, GD2, HER-2, CD30, EGFR, FAP, CD33, CD123, PD-L1, IGF1R, CSPG4, or B7-H4. Further tumor-specific antigens are described, by way of non-limiting example, in US2013/0189268; WO 1999024566 A1; U.S. Pat. No. 7,098,008; and WO 2000020460, each of which is incorporated herein by reference in its entirety.

Therefore, contemplated CARs will generally have a structure of an extracellular binding domain that is (directly) coupled to a hinge domain, which is (directly) coupled to a transmembrane domain, which is (directly) coupled to a (e.g., FcεRIγ) signaling domain. In still further contemplated aspects, contemplated CARs may also include one or more signaling domains in addition to or replacing the FcεRIγ signaling domain, and especially contemplated signaling domains include CD3ζ signaling domains, 4-1BB signaling domains, and CD28 signaling domains. For example, contemplated CAR polypeptides may therefore include any one of the binding domains having SEQ ID NO:41 (EGFR scFv—amino acid sequence; encoded by a nucleic having SEQ ID NO:40, or a sequence having at least 90%, or at least 95% or at least 96%, or at least 97%, or at least 98%, or at least 99% identity to SEQ ID NO:40) and SEQ ID NO:43 (HER2/neu scFv—amino acid sequence; encoded by a nucleic having SEQ ID NO:42, or a sequence having at least 90%, or at least 95% or at least 96%, or at least 97%, or at least 98%, or at least 99% identity to SEQ ID NO:42) that is coupled to a hinge domain (e.g., CD8 hinge as in SEQ ID NO:3), which is in turn coupled to a transmembrane domain (e.g., CD28 TM as in SEQ ID NO:4), which is coupled to a signaling domain (e.g., FcεRIγ signaling domain as in SEQ ID NO:1, CD28 signaling domain as in SEQ ID NO:5, 4-1BB signaling domain as in SEQ ID NO:6, and/or CD3ζ signaling domain as in SEQ ID NO:7)

With respect to the construction of contemplated CARs it should be recognized that CARs can be engineered in numerous manners as described, for example, in WO 2014/039523; US 2014/0242701; US 2014/0274909; US 2013/0280285 and WO 2014/099671, each of which is incorporated herein by reference in its entirety.

Viewed from a different perspective, contemplated CARs target an antigen associated with a specific cancer type, and more particularly cancers that overexpress EGFR and/or HER2 such as lung cancer (e.g., small cell) and breast cancer (e.g., TBNC), thyroid cancer, esophageal cancer, gastric cancer, gastroesophageal cancer, head and neck cancer, etc.

In still further contemplated aspects, NK cells may be further genetically modified to express one or more cytokines, and especially an autocrine growth stimulating cytokine to so provide a selection marker where the cytokine and the CAR are encoded on the same recombinant nucleic acid and/or to render the recombinant cells independent of exogenous IL-2. Therefore, in some embodiments, NK-92 cells are modified to express at least one cytokine. In particular, the at least one cytokine is IL-2, IL-12, IL-15, IL-18, IL-21, or a variant thereof. In preferred embodiments, the cytokine is IL-2 or a variant thereof and especially preferred variants include endoplasmic retention signals (e.g., human IL-2 polypeptide as in SEQ ID NO:9, optionally with ER retention signal as in SEQ ID NO:10). For example, the IL-2 gene is cloned and expressed with a signal sequence that directs the IL-2 to the endoplasmic reticulum. This permits expression of IL-2 at levels sufficient for autocrine activation, but without releasing IL-2 extracellularly (e.g., Exp Hematol. 2005 February; 33(2):159-64.) Alternatively, expression of a cytokine (and especially IL-15) may also be such that the cytokine will be expressed in sufficient quantities to provide an autocrine growth signal to the recombinant cells, but also to allow at least some of the expressed IL-15 to be released from the cell, which will so provide an immune stimulatory signal. For example, such expression may be achieved using a human IL-15 sequence that includes both the signal peptide and an endoplasmic retention sequence. An exemplary DNA and protein sequence for an endoplasmic retained IL-15 is shown in SEQ ID NO:38 and SEQ ID NO:39, respectively.

Where desired, contemplated cells may also express a suicide gene. The term “suicide gene” refers to a transgene that allows for the negative selection of cells expressing the suicide gene. A suicide gene is used as a safety system, allowing cells expressing the gene to be killed by introduction of a selective agent. This is desirable in case the recombinant gene causes a mutation leading to uncontrolled cell growth, or the cells themselves are capable of such growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene. Typically, the suicide gene encodes for a protein that has no ill effect on the cell but, in the presence of a specific compound, will kill the cell. Thus, the suicide gene is typically part of a system.

In one embodiment, the suicide gene is active in NK-92 cells. In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir. In another embodiment, the suicide gene is cytosine deaminase, which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood. 1998 Jul. 15; 92(2):672-82. In a further embodiment, the suicide gene is cytochrome P450, which is toxic in the presence of ifosfamide or cyclophosphamide. See, e.g. Touati et al. “A suicide gene therapy combining the improvement of cyclophosphamide tumor cytotoxicity and the development of an anti-tumor immune response.” Curr Gene Ther. 2014; 14(3):236-46. In yet another embodiment, the suicide gene is iCasp9. Di Stasi, (2011) “Inducible apoptosis as a safety switch for adoptive cell therapy.” N Engl J Med 365: 1673-1683. See also Morgan, “Live and Let Die: A New Suicide Gene Therapy Moves to the Clinic” Molecular Therapy (2012); 20: 11-13. iCasp9 induces apoptosis in the presence of a small molecule, AP1903. AP1903 is biologically inert small molecule, that has been shown in clinical studies to be well tolerated, and has been used in the context of adoptive cell therapy.

Where the modified NK cells are further engineered to express IL-12, it is generally preferred that the IL-12 is expressed as a single chain heterodimer in which the p35 and p40 components are linked together by a flexible linker (in either orientation, p35-linker-p40 or p40-linker-p35). Moreover, it is generally preferred that the heterodimer will be secreted and as such may include a signal peptide for protein export. Therefore, suitable IL-12 sequences as contemplated herein may comprise a nucleic acid sequence with at least 80%, 85%, 90%, 91%, 92%, 93%, 94%, 95%, 96%, 97%, 98% or 99% identity to SEQ ID NO:26 (p35 n.t. sequence), or SEQ ID NO:28 (p40 n.t. sequence). The IL-12 contemplated herein may also comprise an amino acid sequence with at least 80%, 85%, 90%, 91%, 92%, 93%, 94%, 95%, 96%, 97%, 98% or 99% identity to SEQ ID NO:27 (p35 a.a. sequence, isoform 1 precursor), or SEQ ID NO:29 (p40 a.a. sequence, precursor). Thus, the IL-12 single chain p40_p35 sequence may comprise a polypeptide sequence with at least 80%, 85%, 90%, 91%, 92%, 93%, 94%, 95%, 96%, 97%, 98% or 99% identity to SEQ ID NO:31, or may comprise an polynucleotide sequence with at least 80%, 85%, 90%, 91%, 92%, 93%, 94%, 95%, 96%, 97%, 98% or 99% identity to SEQ ID NO:32. Most preferably, but not necessarily, the nucleic acid encoding the IL-12 single chain heterodimer will be part of a polycistronic nucleic acid sequence (e.g., present as a quadracistronic sequence with CAR, CD16, and erIL-2).

Where the modified NK cells are further engineered to express a TGF-beta trap, it is generally preferred that the TGF-beta trap is a single chain dimer of the extracellular domain of a TGFβRII molecule, and most preferably comprises a single chain dimer of the TGF-beta Receptor II ectodomain. Suitable TGF-beta traps are therefore encoded by a polynucleotide sequence with at least 80%, 85%, 90%, 91%, 92%, 93%, 94%, 95%, 96%, 97%, 98% or 99% identity to SEQ ID NO:34 (TGFBRII extracellular domain), or SEQ ID NO:36 (TGF beta trap sequence). The TGF-beta trap contemplated herein may also comprise an amino acid sequence with at least 80%, 85%, 90%, 91%, 92%, 93%, 94%, 95%, 96%, 97%, 98% or 99% identity to SEQ ID NO:35 (TGFBRII extracellular domain), or SEQ ID NO:37 (TGF-beta trap sequence). Other suitable TGF-beta traps include those described in Mol. Canc. Ther. 2012, Vol 11(7), 1477-1487. Most preferably, but not necessarily, the nucleic acid encoding the TGF-beta trap will be part of a polycistronic nucleic acid sequence (e.g., present as a quadracistronic sequence with CAR, CD16, and erIL-2).

Where the modified NK cells are further engineered to express a homing receptor it is noted that the term “homing receptor” refers to a receptor that activates a cellular pathway that results directly or indirectly in the cell migrating toward a target cell or tissue. For example, homing receptors expressed by leukocytes are used by leukocytes and lymphocytes to enter secondary lymphoid tissues via high endothelial venules. Homing receptors can also be used by cells to migrate toward the source of a chemical gradient, such as a chemokine gradient. Examples of homing receptors include G-protein coupled receptors such as chemokine receptors, including CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCXCKR, D6, and DARC; cytokine receptors; cell adhesion molecules such as selectins, including L-selectin (CD62L), integrins such as α4β7 integrin, LPAM-1, and LFA-1. Homing receptors generally bind to cognate ligands on the target tissues or cell. In some embodiments, homing receptors bind to addressins on the endothelium of venules, such as mucosal vascular addressing cell adhesion molecule 1 (MAdCAM-1).

In some exemplary embodiments, the chemokines and homing receptors contemplated herein may comprise a polypeptide sequence or a polynucleotide sequence having at least 80%, 85%, 90%, 91%, 92%, 93%, 94%, 95%, 96%, 97%, 98% or 99% identity to SEQ ID NO:13 (CCR7 n.t. sequence), or SEQ ID NO:14 (CCL19 n.t. sequence), or SEQ ID NO:15 (CCL21 n.t. sequence), or SEQ ID NO:16 (CXCR2 n.t. sequence), or SEQ ID NO:17 (CXCR2 a.a. sequence), or SEQ ID NO:18 (CXCL14 n.t. sequence), or SEQ ID NO:19 (CXCL4 a.a. sequence), or SEQ ID NO:20 (CD62L n.t. sequence), or SEQ ID NO:21 (CD62L a.a. sequence), or SEQ ID NO:22 (IL-8 n.t. sequence), or SEQ ID NO:23 (IL-8 a.a. sequence), or SEQ ID NO:24 (CXCL1 n.t. sequence), or SEQ ID NO:25 (CXCL1 a.a. sequence). Most preferably, but not necessarily, the nucleic acid encoding the homing receptor will be part of a polycistronic nucleic acid sequence (e.g., present as a quadracistronic sequence with CAR, CD16, and erIL-2).

Of course, it should be noted that all of the recombinant proteins can be expressed from individual recombinant sequences. However, it is generally preferred that where multiple recombinant sequences are expressed (e.g., CAR, CD16, cytokine, TGF-beta trap), coding regions may be arranged in a polycistronic unit with at least two or at least three or at least four coding regions encoding the recombinant proteins. Therefore, transgenes can be engineered into an expression vector by any mechanism known to those of skill in the art. Where multiple transgenes are to be inserted into a cell, transgenes may be engineered into the same expression vector or a different expression vector. In some embodiments, the cells are transfected with mRNA encoding the transgenic protein to be expressed. In some embodiments, the cells are transfected with DNA encoding the transgenic protein to be expressed. Transgenes, mRNA and DNA can be introduced into the NK-92 cells using any transfection method known in the art, including, by way of non-limiting example, infection, viral vectors, electroporation, lipofection, nucleofection, or “gene-gun.”

In preferred embodiments, it should therefore be noted that the genetically modified NK cell (especially where the cell expresses a CAR and CD16 or variant thereof) will exhibit three distinct modes of cell killing: General cytotoxicity which is mediated by activating receptors (e.g., an NKG2D receptor), ADCC which is mediated by antibodies bound to a target cell, and CAR mediated cytotoxicity. Where desired, therapeutic effect in the tumor microenvironment may be further increased via expression and secretion of IL-12 (e.g., as a single chain heterodimer), via expression and presentation/secretion of a TGF-beta trap (e.g., as a single chain dimer of the TGF-beta Receptor II ectodomain) or via expression of a homing receptor (e.g., CCR7). As will be readily apparent, contemplated genetically modified cells can be used for treatment of various diseases, and especially of various cancers and viral infections where a diseased cell presents a disease-specific or disease-associated antigen. Consequently, the inventors contemplate methods of treating patients with modified NK or NK-92 cells as described herein. In one embodiment, the patient is suffering from cancer (e.g., a tumor) and the modified NK-92 cell or cell line expresses a CAR specific for an antigen expressed on the surface of a cell from the cancer or tumor. As noted above, in some embodiments, the cancer is lung cancer, a breast cancer, a thyroid cancer, an esophageal cancer, a gastric cancer, a gastroesophageal cancer, or a head and neck cancer.

Contemplated modified NK or NK-92 cells can be administered to an individual by absolute numbers of cells. For example, the individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) modified NK-92 cells per injection, or any ranges between any two of the numbers, end points inclusive. In other embodiments, modified NK-92 cells can be administered to an individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) modified NK-92 cells per kilogram of the individual, or any ranges between any two of the numbers, end points inclusive. In other embodiments, the total dose may be calculated by m² of body surface area, including about 1×10¹¹, 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion NK-92 cells are administered to a patient.

Modified NK-92 cells, and optionally other anti-cancer agents can be administered once to a patient with cancer or infected with a virus or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In one embodiment, where the modified NK cells express a suicide gene, the patient is administered an agent to trigger modified NK cell death. In one embodiment, the agent is administered at a time point after administration of the modified NK cells that is sufficient for the NK cells to kill target cells.

In one embodiment, the modified NK cells are irradiated prior to administration to the patient. Irradiation of NK cells is described, for example, in U.S. Pat. No. 8,034,332, which is incorporated herein by reference in its entirety. In one embodiment, modified NK cells that have not been engineered to express a suicide gene are irradiated.

Furthermore, it should be appreciated that contemplated treatments will also include administration of other immune therapeutic entities, and especially preferred immune therapeutic entities include a viral cancer vaccine (e.g., adenoviral vector encoding cancer specific antigens), a bacterial cancer vaccine (e.g., non-pyrogenic E. coli expressing one or more cancer specific antigens), a yeast cancer vaccine, N-803 (also known as ALT-803, ALTOR Biosciences), an antibody (e.g., binding to a tumor associated antigen or patient specific tumor neoantigen), a stem cell transplant (e.g., allogeneic or autologous), and a tumor targeted cytokine (e.g., NHS-IL12, IL-12 coupled to a tumor targeting antibody or fragment thereof).

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before aspects of the present inventive subject matter are disclosed and described in more detail, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1: HER2.CAR t-haNK Cells

Unless specified otherwise in the following examples, the inventors used NK-92 cells for all transfections of the cells with recombinant nucleic acids. Furthermore, and also unless noted otherwise, all recombinant nucleic acids were linearized DNA constructs encoding a tri- or quadracistronic configuration.

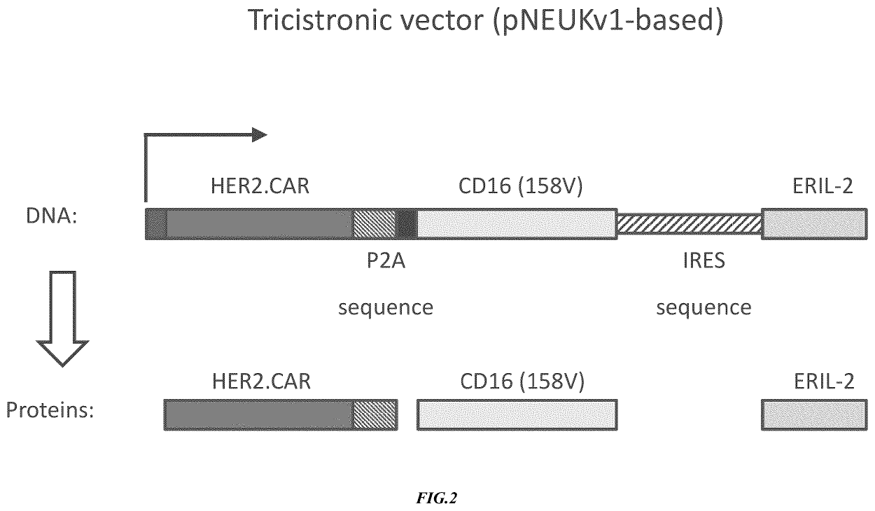

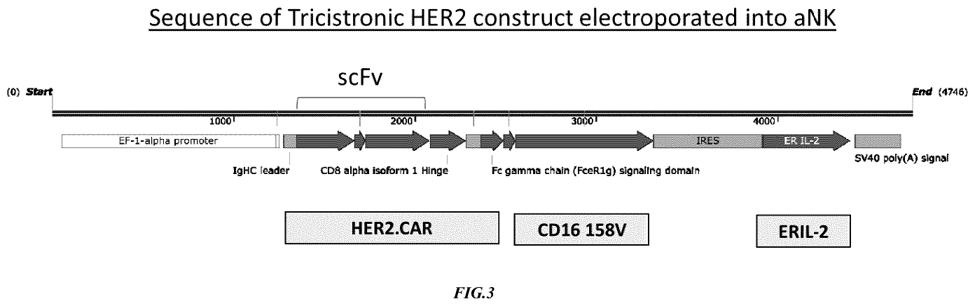

To generate HER2.CAR t-haNK cells, a recombinant DNA molecule was assembled as is schematically depicted in FIG. 2 where the tricistronic configuration included a sequence encoding a HER2.CAR followed by a P2A sequence, which was followed by a sequence encoding CD16 (or CD16^{158V}), and which in turn was followed by an IRES sequence element upstream of a sequence encoding erIL-2. Unless otherwise noted, the HER2.CAR had a structure as exemplarily shown in FIG. 1 (however, with an Fc epsilon signaling sequence) and had a nucleic acid sequence of SEQ ID NO:30. Of course, it should be noted that other suitable sequences for the production of HER2.CAR may have a sequence identity of at least 90%, or at least 95%, or at least 96%, or at least 97%, or at least 98%, or at least 99% to SEQ ID NO:30. The primary transcript of the tricistronic nucleic acid then lead to the formation of HER2.CAR, CD16^{158V}, and erIL-2 as the recombinant polypeptides. FIG. 3 exemplarily and schematically depicts a linearized recombinant nucleic acid used for transfection of the NK-92 cells.

All transfections generally followed standard protocol: NK-92 cells were grown in X-Vivo10 medium (Lonza, Basel, Switzerland) supplemented with 5% Human AB Serum (Valley Biomedical, Winchester, VA) and 500 IU/mL IL-2 (Prospec, Rehovot, Israel). Cells were electroporated with tricistronic or quadracistronic DNA using the Neon™ electroporation device (Life Technologies, Carlsbad, CA), following the manufacturer's parameters for NK-92 cells (1250 V, 10 ms, 3 pulses) and using 5 μg of DNA per 10⁶ cells in a volume of 100 μl. Electroporated cells were transferred into medium (same as above) without exogenously added IL-2.

Selection for recombinant cells and clones was based on continued cell culture in the absence of exogenously added IL-2 as all recombinant cells included a recombinant autocrine growth promoting cytokine (e.g., IL-2, erIL-2, IL-15, or erIL-15).

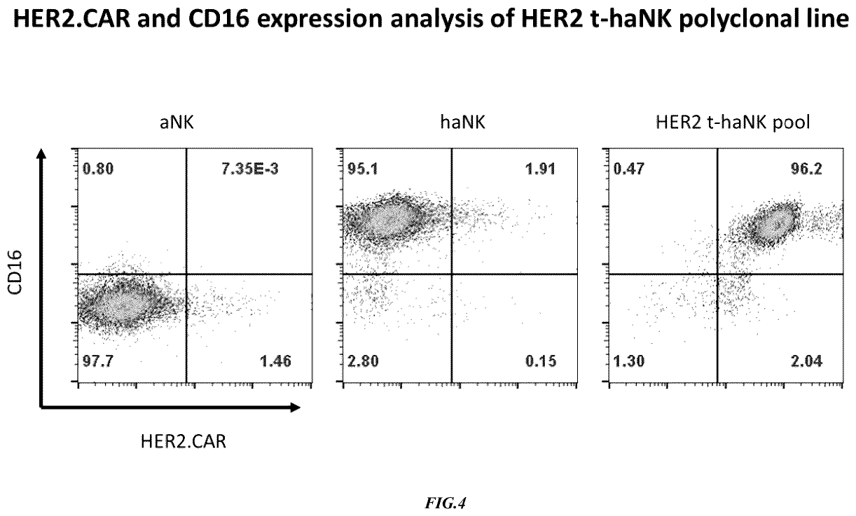

The HER2.CAR and CD16 expression on the NK-92 cell surface was determined by flow cytometry using biotinylated soluble HER2 protein and APC-labeled streptavidin, and fluorescently labeled anti-CD16 antibody. Exemplary results for HER2.CAR and CD16 expression (polyclonal) are shown in FIG. 4 along with aNK (not expressing CD16) and haNK (expressing CD16) controls. As can be readily seen from FIG. 4, the polyclonal cell cultures had a significant and strong expression for both, HER2.CAR and CD16^{158V}. In this context, it should be noted that the HER2.CAR was expressed at notably higher levels where the HER2.CAR had a FcεRIγ signaling portion as compared to other signaling portions (data not shown).

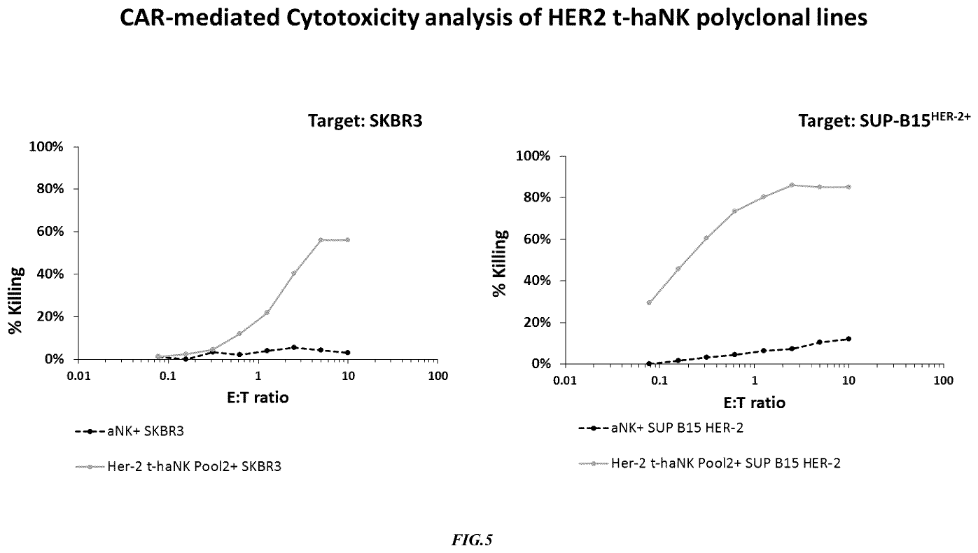

HER2.CAR t-haNK cells had strong and target specific CAR-mediated cytotoxicity as can be seen from the results depicted in FIG. 5. Here, SKBR3 cells and SUP-B15^{HER-2+} cells were co-incubated with HER2.CAR t-haNK cells at varying effector to target cell ratios (e.g., between 0.075 and 10) in a flow-cytometry based in vitro cytotoxicity assay, and target specificity was compared to aNK cells as indicated. As can be seen, CAR-mediated cytotoxicity using a polyclonal HER2.CAR t-haNK cell population was >60% for SKBR3 cells and approaching 90% for SUP-B15^{HER-2+} cells.

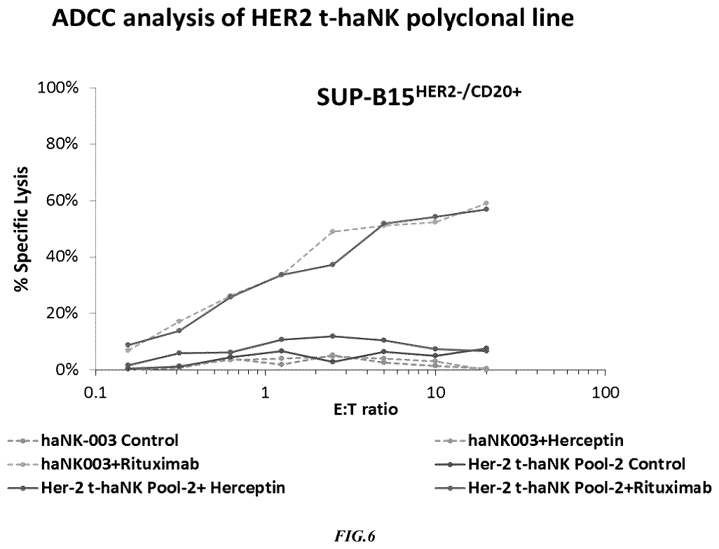

In a similar manner, ADCC was tested using SUP-B15^{HER-2-/CD20+} cells and rituximab as target specific antibody and Herceptin as control antibody. Once again, the polyclonal HER2.CAR t-haNK cells had strong and target specific ADCC approaching 60% as can be seen from the exemplary results depicted in FIG. 6, with no substantial off-target toxicity.

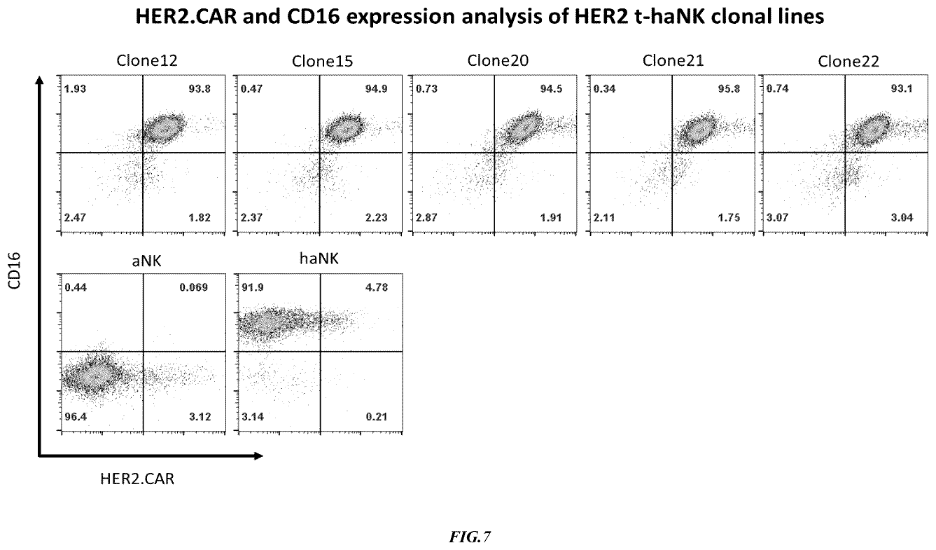

A number of individual clones from the HER2.CAR t-haNK cell population were then prepared following dilution propagation, and expression analysis was once more performed via FACS using the same procedure as described above. aNK and haNK cells were used as controls. Once more, it was observed that all individual clones had a significant and strong expression of both HER2.CAR and CD16^{158V} as can be seen from the exemplary results of FIG. 7.

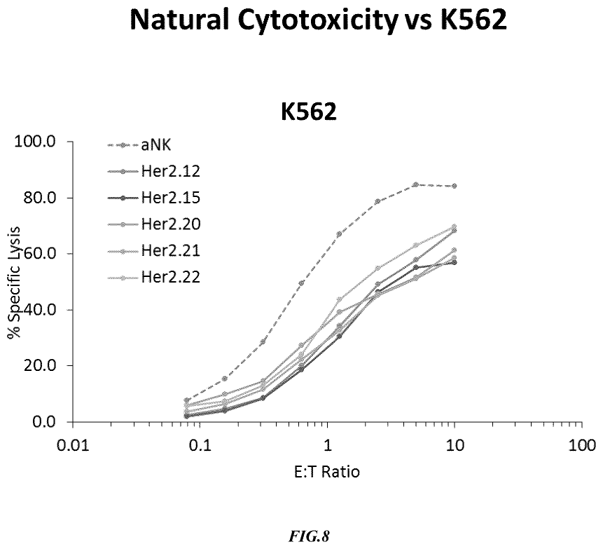

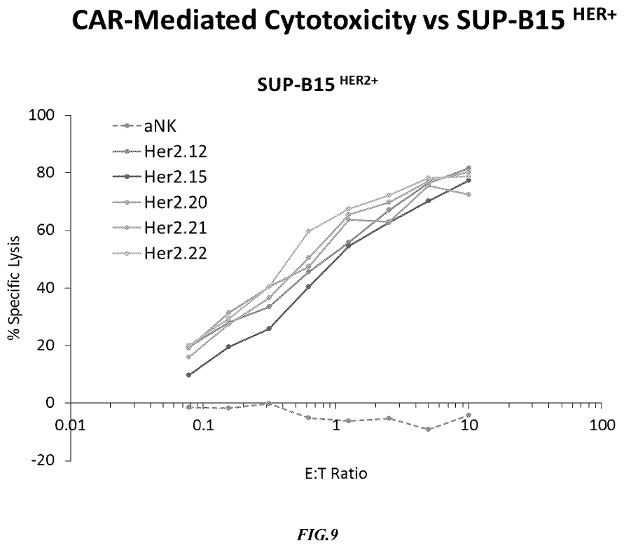

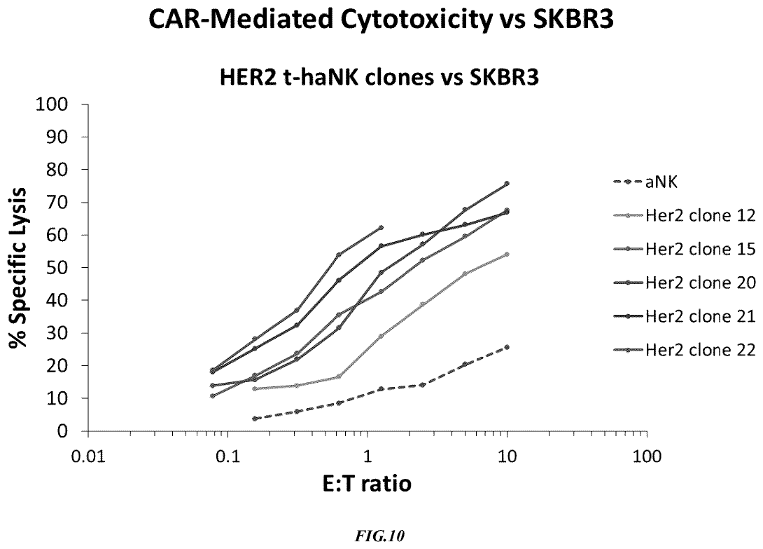

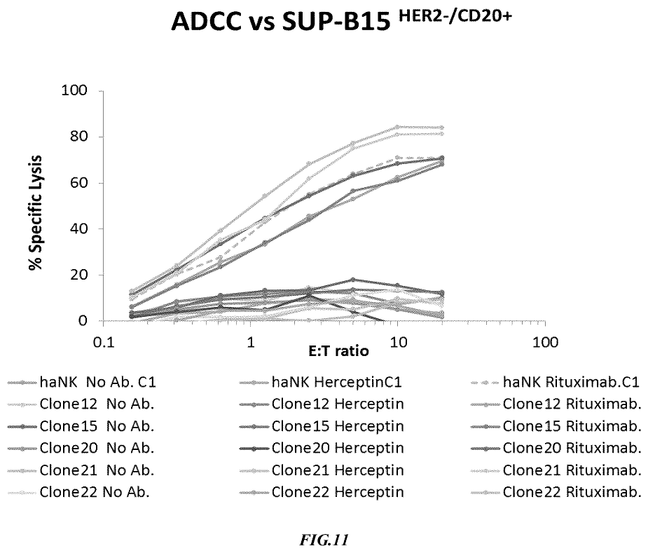

Individual HER2.CAR t-haNK cell clones were tested for natural cytotoxicity using K562 cells and results were compared to non-transfected aNK cells as is shown in FIG. 8. Here, specific lysis at effector to target cell ratios as indicated was lower than control aNK lysis, however, only about 10-20% less. On the other hand, CAR-mediated cytotoxicity was once more substantial and target restricted using SUP-B15^{HER-2+} cells and exemplary results for specific HER2.CAR t-haNK cell clones are shown in FIG. 9 over a wide range of effector to target ratios. Likewise, where SKBR3 cells were used as target cells, notable CAR-mediated cytotoxicity was again observed for all of the tested HER2.CAR t-haNK cell clones as is exemplarily shown in FIG. 10. Selected HER2.CAR t-haNK cell clones were also tested for ADCC against SUP-B15^{HER-2-/CD20+} cells using rituximab as target specific antibody and Herceptin as control antibody. As can be taken from FIG. 11, all HER2.CAR t-haNK cell clones had once more significant, strong, and target specific ADCC against control.

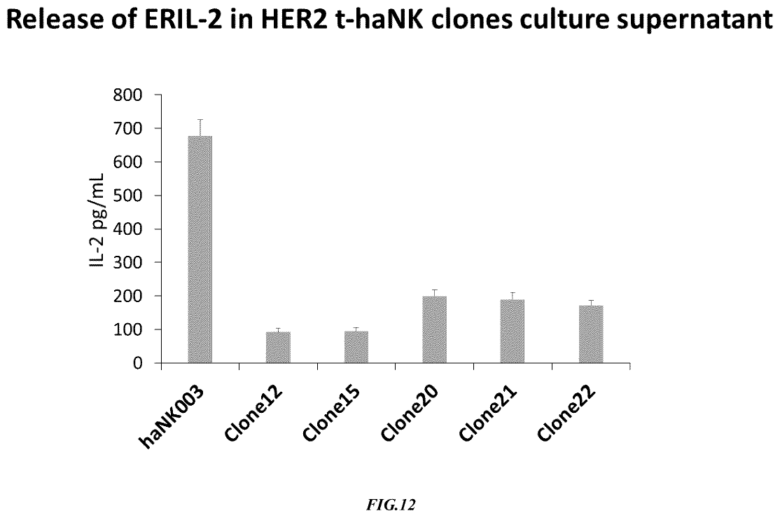

While all HER2.CAR t-haNK polyclonal cultures and cell clones propagated well in the absence of exogenous IL-2, expression of er-IL2 was also tested along with the quantity of erIL-2 released in the culture medium. Exemplary results are shown in FIG. 12 depicting a minor release of erIL-2 into the culture medium. Notably, the extracellular erIL-2 release was significantly less than that of a closely related haNK003 cell line as is shown in FIG. 12. Such reduced release may further advantageously reduce potential systemic effects otherwise attributable to IL-2 (e.g., vascular permeability, systemic vascular leak, etc.).

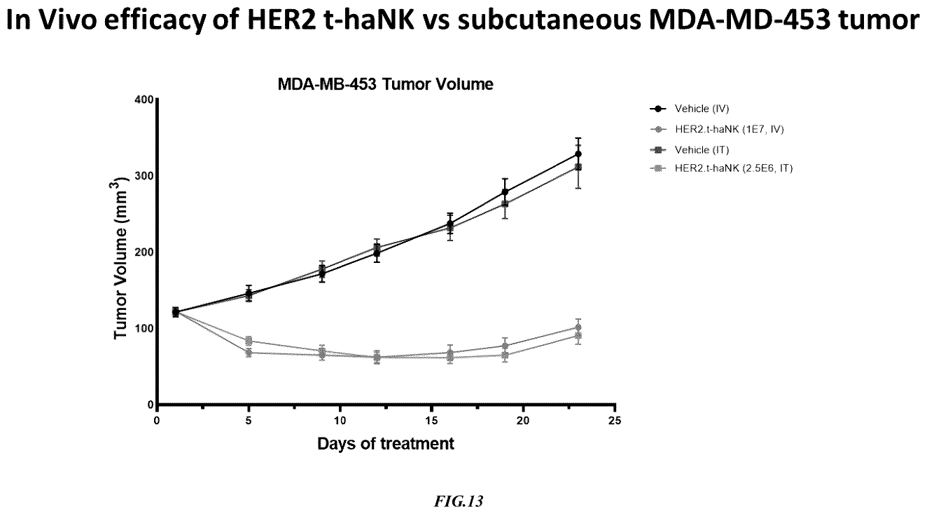

To test in vivo efficacy of the HER2.CAR t-haNK cells, NSG mice (NOD.Cg-Prkdc^{scid}Il2rg^{tm1Wjl}/SzJ) were implanted with MDA-MB-453 tumor cells subcutaneously, and tumor volume was measured. When tumors reached an average ˜120 mm³, HER2.CAR t-haNK cells were administered intratumorally or intravenously twice a week for the duration of the study. Following administration of HER2.CAR t-haNK cells, tumor volume was sustainably reduced compared to vehicle control treatment as is demonstrated by the exemplary results in FIG. 13. It should be appreciated that intratumoral administration required significantly less HER2.CAR t-haNK cells for the same reduction in tumor volume than systemic intravenous administration.

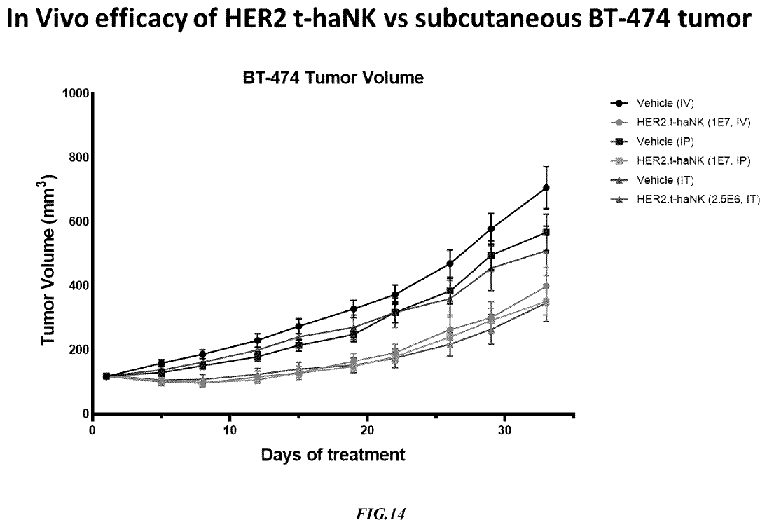

Similarly, in vivo efficacy of the HER2.CAR t-haNK cells was tested in NSG mice that were implanted with BT-474 tumor cells subcutaneously, and the tumor volume was measured over time. When tumors reached an average ˜120 mm³, HER2.CAR t-haNK cells were administered intratumorally or intravenously twice a week for the duration of the study. Following administration of HER2.CAR t-haNK cells, the increase in tumor volume was substantially reduced over the time period tested as is shown by the exemplary results in FIG. 14. Once more, it should be appreciated that intratumoral administration required significantly less HER2.CAR t-haNK cells for the same reduction in tumor growth than systemic intravenous administration.

### Example 2: EGFR.CAR t-haNK Cells

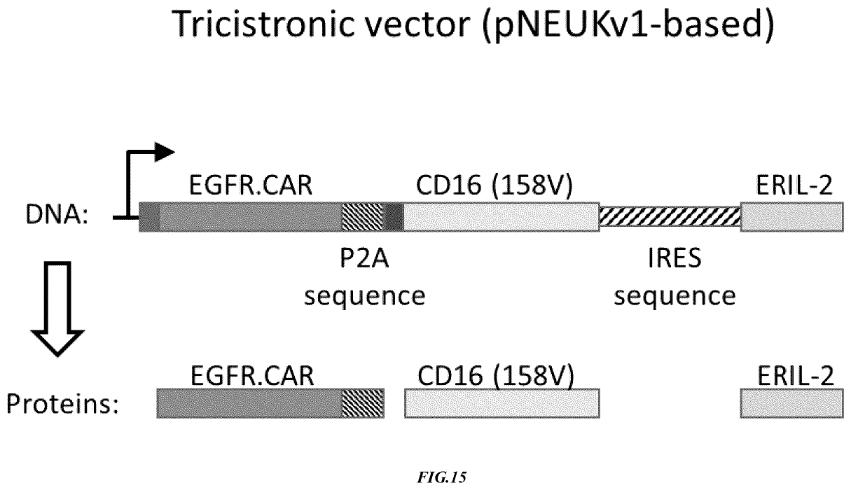

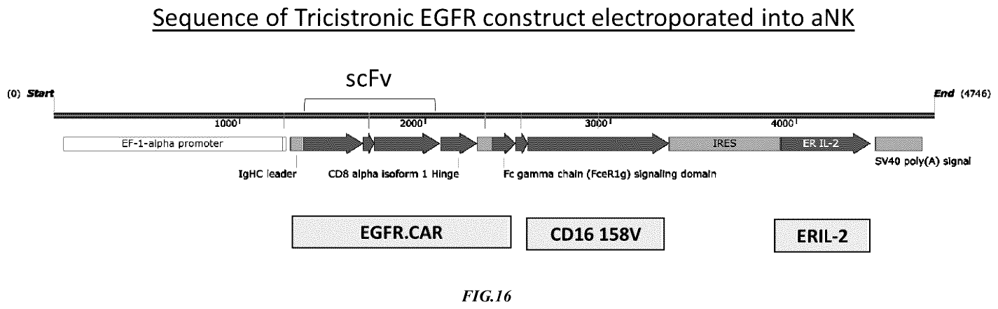

To generate EGFR.CAR t-haNK cells, a recombinant DNA molecule was assembled as is schematically depicted in FIG. 15 where the tricistronic configuration included a sequence encoding a EGFR.CAR followed by a P2A sequence, which was followed by a sequence encoding CD16 (or CD16^{158V}), and which in turn was followed by an IRES sequence element upstream of a sequence encoding erIL-2. Unless otherwise noted, the EGFR.CAR had a structure as exemplarily shown in FIG. 1 (however, with an Fc epsilon signaling sequence) and had a nucleic acid sequence of SEQ ID NO:33. Of course, it should be noted that other suitable sequences for the production of EGFR.CAR may have a sequence identity of at least 90%, or at least 95%, or at least 96%, or at least 97%, or at least 98%, or at least 99% to SEQ ID NO:33. The primary transcript of the tricistronic nucleic acid then lead to the formation of EGFR.CAR, CD16^{158V}, and erIL-2 as the recombinant polypeptides. FIG. 16 exemplarily and schematically depicts a linearized recombinant nucleic acid used for transfection of the NK-92 cells.

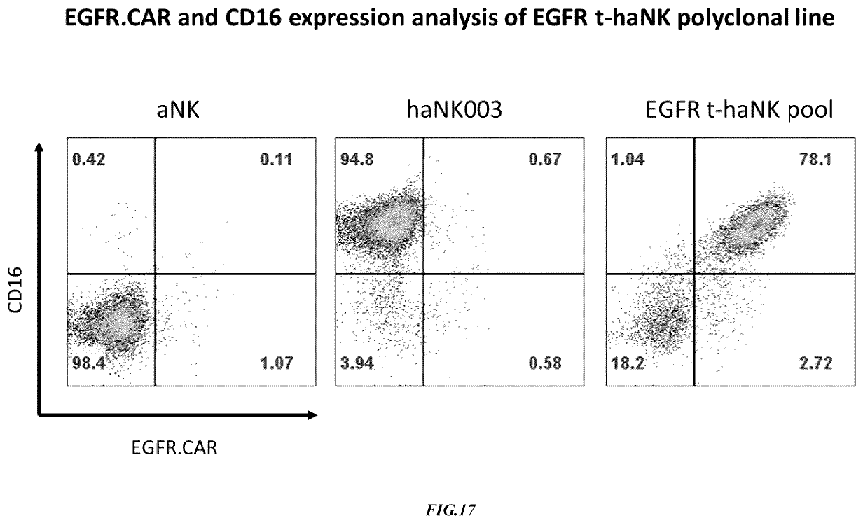

The EGFR.CAR and CD16 expression on the NK-92 cell surface was determined by flow cytometry using biotinylated anti-scFv antibody and APC-labeled streptavidin, and fluorescently labeled anti-CD16 antibody. Exemplary results for EGFR.CAR and CD16 expression (polyclonal) are shown in FIG. 17 along with aNK (not expressing CD16) and haNK (expressing CD16) controls. As can be readily seen from FIG. 17, the polyclonal cell cultures had a significant and strong expression for both, EGFR.CAR and CD16^{158V}. In this context, it should once more be noted that the EGFR.CAR was expressed at notably higher levels where the HER2.CAR had a FcεRIγ signaling portion as compared to other signaling portions (data not shown).

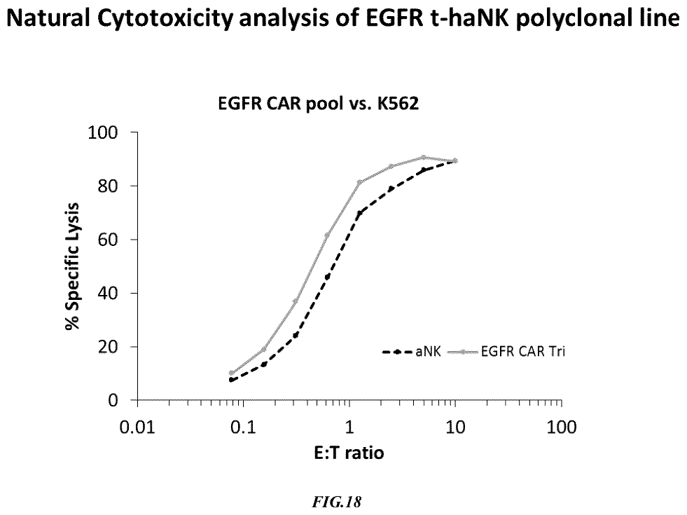

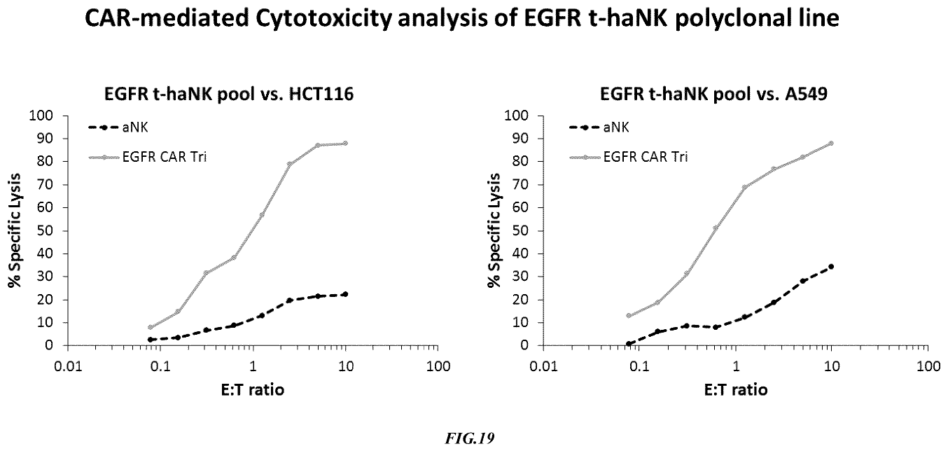

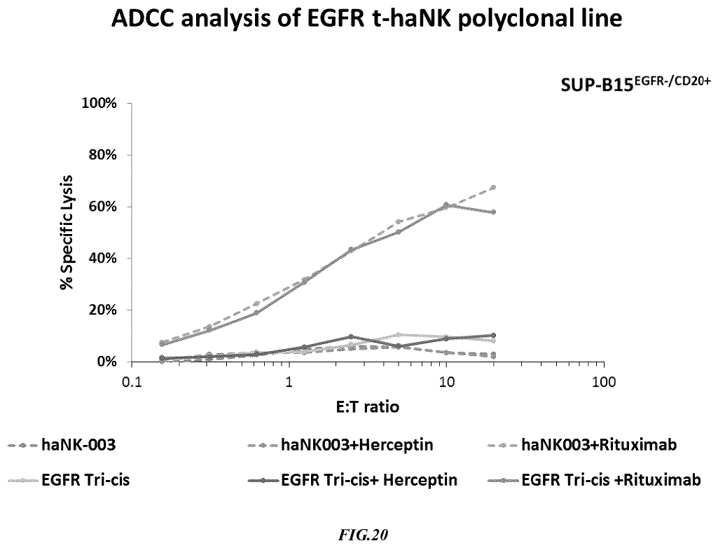

Natural cytotoxicity was determined for polyclonal cell culture of the EGFR.CAR t-haNK cells, and FIG. 18 depicts an exemplary set of results. Here, the natural cytotoxicity against K562 cells indeed exceeded the cytotoxicity of aNK control cells. Similarly, CAR-mediated cytotoxicity was determined for polyclonal cell culture of the EGFR.CAR t-haNK cells against HCT116 target cells and against A549 cells, both of which expressed EGFR. As can be seen from the graphs in FIG. 19, CAR-mediated cytotoxicity was significant and specific. In still further experiments, the inventors tested EGFR.CAR t-haNK polyclonal cells for ADCC against SUP-B15^{EGFR-/CD20+} cells using rituximab as target specific antibody and Herceptin as control antibody. As can be taken from FIG. 20, all EGFR.CAR t-haNK cells had once more significant, strong, and target specific ADCC versus control.

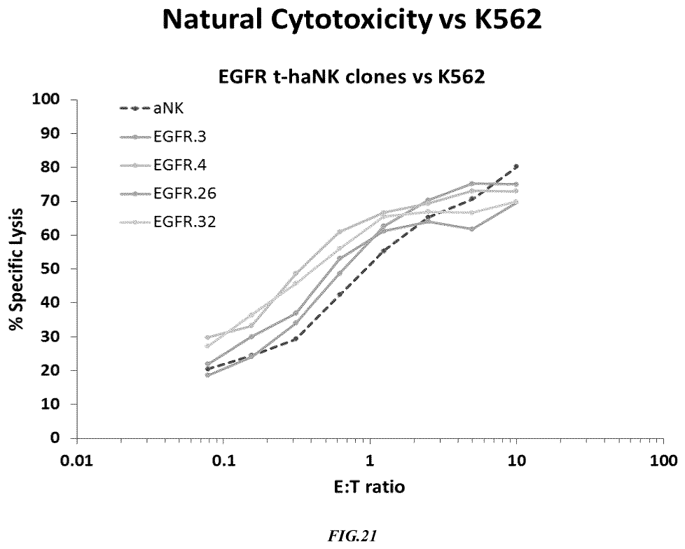

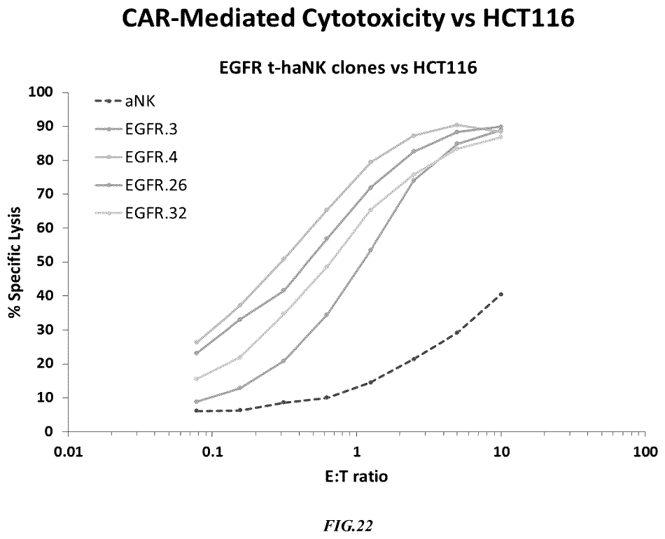

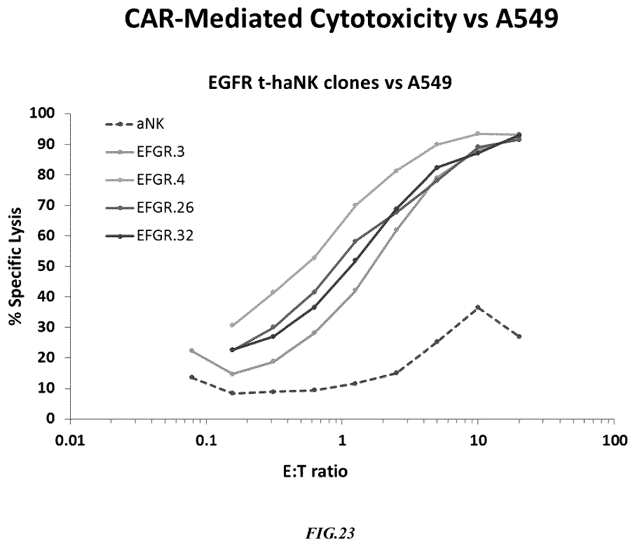

After dilution propagation, selected EGFR.CAR t-haNK cell clones were tested for natural cytotoxicity against 562 cells. Notably, all selected EGFR.CAR t-haNK cell clones had substantially identical natural cytotoxicity which was comparable to the aNK control as can be taken from FIG. 21. Selected EGFR.CAR t-haNK cell clones were then tested for CAR-mediated cytotoxicity against HCT116 tumor cells, and exemplary results are shown in the graphs of FIG. 22. Once more all EGFR.CAR t-haNK cell clones had very significant CAR-mediated cytotoxicity against HCT116 tumor cells approaching 90% specific lysis. Similar results were obtained using A549 tumor cells as target cells as is shown in FIG. 23.

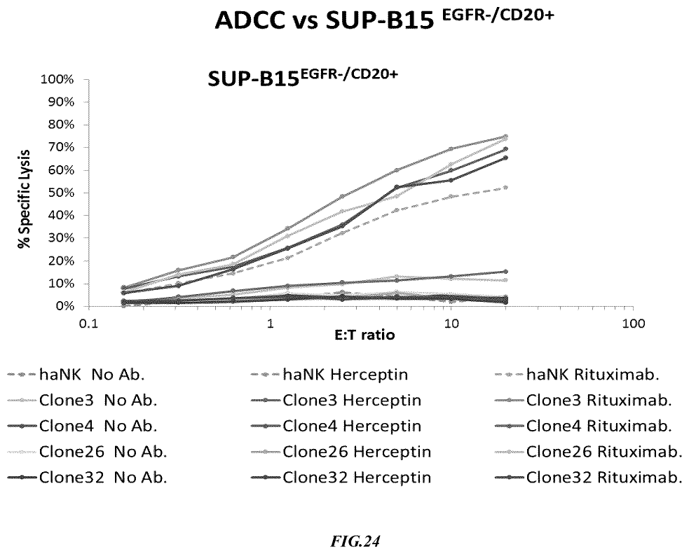

In still further experiments, the inventors determined ADCC for selected EGFR.CAR t-haNK cell clones using SUP-B15^{EGFR-/CD20+} cells as target cells, rituximab as target specific antibody, and Herceptin as control antibody. As already observed with the polyclonal cell culture and as can be seen from FIG. 24, selected EGFR.CAR t-haNK cell clones exhibited effective ADCC in the presence of cognate antibodies.

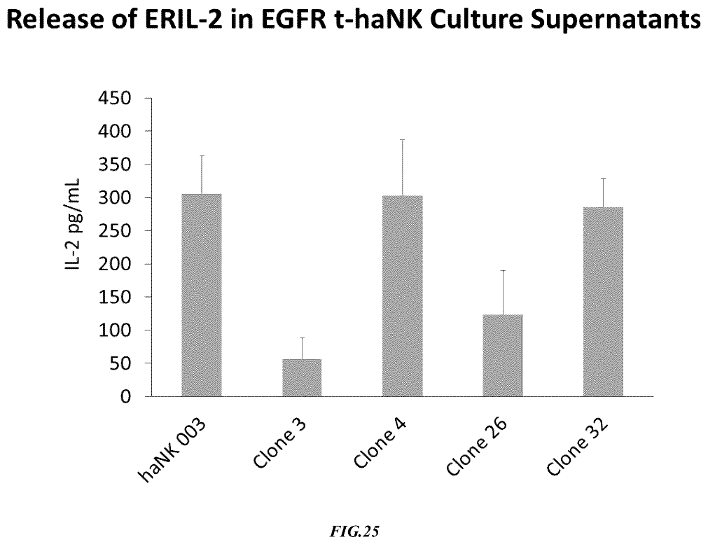

While all EGFR.CAR t-haNK polyclonal cultures and cell clones propagated well in the absence of exogenous IL-2, expression of er-IL2 was also tested along with the quantity of erIL-2 released in the culture medium. Exemplary results are shown in FIG. 25 depicting for some clones a minor release of erIL-2 into the culture medium while other clones had an erIL-2 release comparable to control. As noted above, reduced erIL-2 release may further advantageously reduce potential systemic effects otherwise attributable to IL-2 (e.g., vascular permeability, systemic vascular leak, etc.).

### Example 3: Quadracistronic Constructs

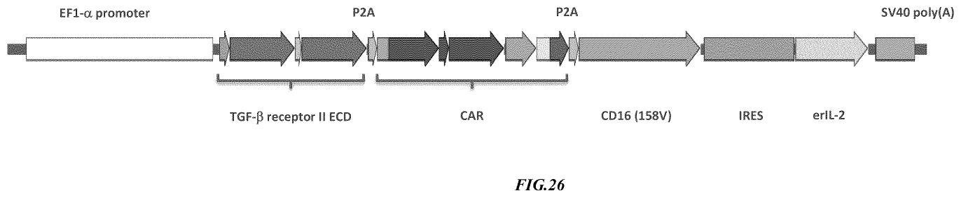

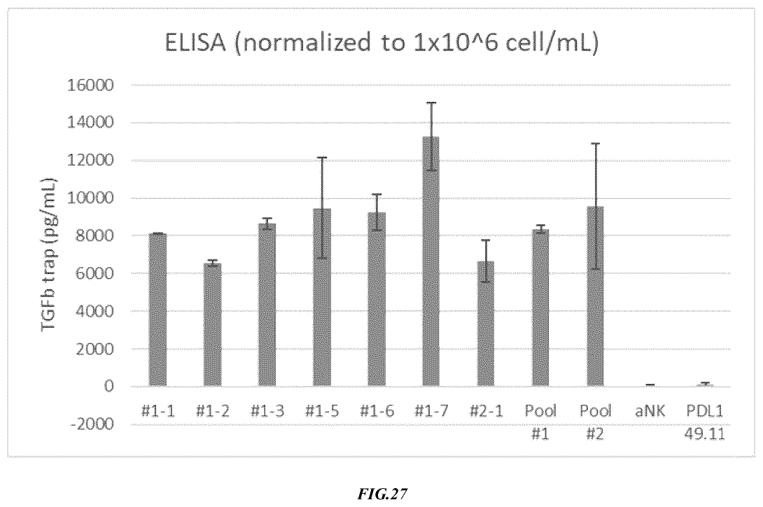

While the above examples were performed with tricistronic constructs it should be appreciated that the same constructs can also be implemented in a quadracistronic construct to express IL-12, a TGF-beta trap, or a homing receptor to so reduce immune suppression in the tumor microenvironment and enrich the tumor microenvironment with thusly modified NK cells. One exemplary quadracistronic construct is schematically illustrated in FIG. 26 where a nucleic acid sequence encoding a TGF-beta trap is upstream of the tricistronic construct as discussed above. As can be seen from FIG. 26, a P2A sequence is located between the TGF-beta trap and the CAR to ensure coordinated expression while producing distinct proteins. Expression of the TGF-beta trap was then tested in an ELISA to ascertain that the quadracistronic construct indeed produced a functional TGF-beta trap, and FIG. 27 depicts exemplary results versus control. Notably, expression levels of the TGF-beta trap were significant throughout all recombinant clones with quadracistronic construct.

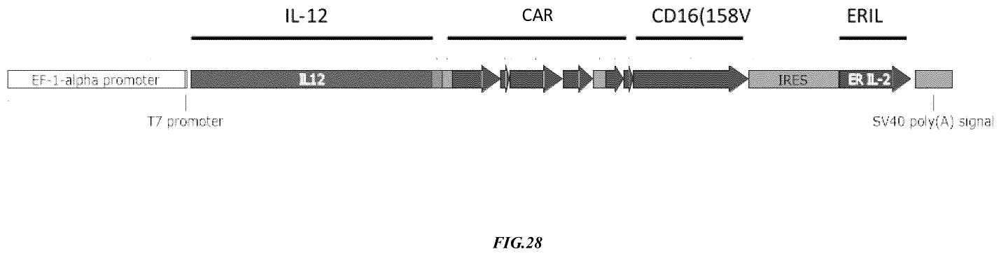

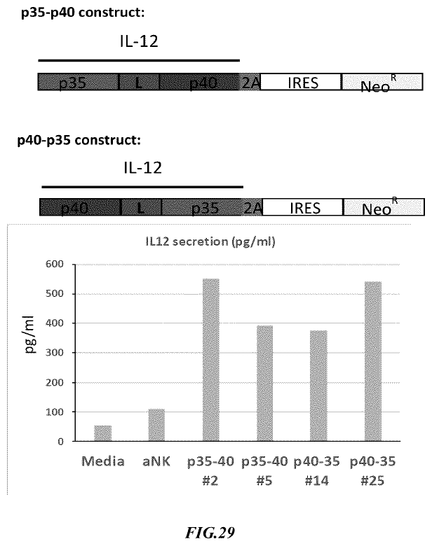

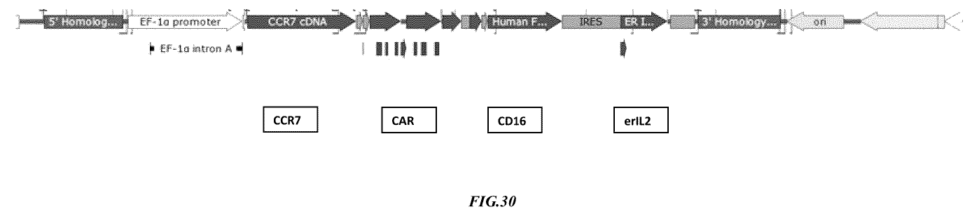

Similarly, quadracistronic constructs were prepared using a secreted IL-12 single chain heterodimer as is schematically depicted in FIG. 28. Once more, a P2A sequence was placed between the IL-12 single chain heterodimer sequence and the CAR sequence. FIG. 29 shows exemplary embodiments of the single chain heterodimer with alternate order of p35 and p40 subunits (which were both separated by a linker sequence). As can be seen from the expression levels tested, both single chain heterodimers expressed equally well in the cells tested relative to control. In a still further embodiment, a quadracistronic construct was prepared using CCR7 as a homing receptor and an exemplary quadricictronic arrangement is depicted in FIG. 30.

Of course, it should be recognized that for all nucleic acid sequences provided herein the corresponding encoded proteins are also expressly contemplated herein. Likewise, for all amino acid sequences, corresponding nucleic acids sequences are also contemplated herein (with any codon usage).

All patent applications, publications, references, and sequence accession numbers cited in the present specification are hereby incorporated by reference in their entirety.

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

It is understood that all numerical values described herein (e.g., pH, temperature, time, concentration, amounts, and molecular weight, including ranges) include normal variation in measurements encountered by one of ordinary skill in the art. Thus, numerical values described herein include variation of +/−0.1 to 10%, for example, +/−0.1%, 0.5%, 1%, 2%, 3%, 4%, 5%, 6%, 7%, 8%, 9%, or 10%. It is to be understood, although not always explicitly stated, that all numerical designations may be preceded by the term “about.” Thus, the term about includes variation of +/−0.1 to 10%, for example, +/−0.1%, 0.5%, 1%, 2%, 3%, 4%, 5%, 6%, 7%, 8%, 9%, or 10% of the numerical value. It is also to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein include the end points of the range, and include all values between the end points of the range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this disclosure.

As used herein, “immunotherapy” refers to the use of NK-92 cells, modified or unmodified, naturally occurring or modified NK cell or T-cell, whether alone or in combination, and which are capable of inducing cytotoxicity when contacting a target cell.

As used herein, “natural killer (NK) cells” are cells of the immune system that kill target cells in the absence of a specific antigenic stimulus, and without restriction according to major histocompatibility complex (MHC) class. Target cells may be tumor cells or cells harboring a virus. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

The term “NK-92” refers to natural killer cells derived from the highly potent unique cell line described in Gong et al. (1994), rights to which are owned by NantKwest (hereafter, “NK-92™ cells”). The immortal NK cell line was originally obtained from a patient having non-Hodgkin's lymphoma. Unless indicated otherwise, the term “NK-92™” is intended to refer to the original NK-92 cell lines as well as NK-92 cell lines that have been modified (e.g., by introduction of exogenous genes). NK-92™ cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92™, NK-92™-CD16, NK-92™-CD16-γ, NK-92™-CD16-ζ, NK-92™-CD16(F176V), NK-92™ MI, and NK-92™ CI. NK-92 cells are known to persons of ordinary skill in the art, to whom such cells are readily available from NantKwest, Inc.

The term “aNK” refers to an unmodified natural killer cells derived from the highly potent unique cell line described in Gong et al. (1994), rights to which are owned by NantKwest (hereafter, “aNK™ cells”). The term “haNK” refers to natural killer cells derived from the highly potent unique cell line described in Gong et al. (1994), rights to which are owned by NantKwest, modified to express CD16 on the cell surface (hereafter, “CD16+NK-92™ cells” or “haNK® cells”). In some embodiments, the CD16+NK-92™ cells comprise a high affinity CD16 receptor on the cell surface. The term “taNK” refers to natural killer cells derived from the highly potent unique cell line described in Gong et al. (1994), rights to which are owned by NantKwest, modified to express a chimeric antigen receptor (hereafter, “CAR-modified NK-92™ cells” or “taNK® cells”). The term “t-haNK” refers to natural killer cells derived from the highly potent unique cell line described in Gong et al. (1994), rights to which are owned by NantkWest, modified to express CD16 on the cell surface and to express a chimeric antigen receptor (hereafter, “CAR-modified CD16+NK-92™ cells” or “t-haNK™ cells”). In some embodiments, the t-haNK™ cells express a high affinity CD16 receptor on the cell surface.

A “modified NK-92 cell” refers to an NK-92 cell that expresses an exogenous gene or protein, such as an Fc receptor, a CAR, a cytokine (such as IL-2 or IL-15), and/or a suicide gene. In some embodiments, the modified NK-92 cell comprises a vector that encodes for a transgene, such as an Fc receptor, a CAR, a cytokine (such as IL-2 or IL-15), and/or a suicide gene. In one embodiment, the modified NK-92 cell expresses at least one transgenic protein.

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a patient, since the time between irradiation and infusion should be no longer than four hours in order to preserve optimal activity. Alternatively, NK-92 cells may be prevented from proliferating by another mechanism.

As used herein, “inactivation” of the NK-92 cells renders them incapable of growth. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of non-limiting example, by administering an inactivating agent to which the NK-92 cells are sensitive.

As used herein, the terms “cytotoxic” and “cytolytic,” when used to describe the activity of effector cells such as NK-92 cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK-92 cells is due to cytolysis.

The term “kill” with respect to a cell/cell population is directed to include any type of manipulation that will lead to the death of that cell/cell population.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCγR) bind to the IgG class of antibodies. FCγRIII-A is a low affinity Fc receptor bind to IgG antibodies and activate ADCC. FCγRIII-A are typically found on NK cells. NK-92 cells do not express FCγRIII-A. Fc-epsilon receptors (FcεR) bind to the Fc region of IgE antibodies.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92 cell is targeted to cells expressing certain antigens on the cell surface, based on the specificity of the scFv domain. The scFv domain can be engineered to recognize any antigen, including tumor-specific antigens and virus-specific antigens. For example, CD19CAR recognizes CD19, a cell surface marker expressed by some cancers.

The term “tumor-specific antigen” as used herein refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell.

The term “virus-specific antigen” as used herein refers to antigens that are present on a virus-infected cell but not detectable on a normal cell derived from the same tissue or lineage as the virus-infected cell. In one embodiment, a virus-specific antigen is a viral protein expressed on the surface of an infected cell.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, “percent identity” refers to sequence identity between two peptides or between two nucleic acid molecules. Percent identity can be determined by comparing a position in each sequence which may be aligned for purposes of comparison. When a position in the compared sequence is occupied by the same base or amino acid, then the molecules are identical at that position. Homologous nucleotide sequences include those sequences coding for naturally occurring allelic variants and mutations of the nucleotide sequences set forth herein. Homologous nucleotide sequences include nucleotide sequences encoding for a protein of a mammalian species other than humans. Homologous amino acid sequences include those amino acid sequences which contain conservative amino acid substitutions and which polypeptides have the same binding and/or activity. In some embodiments, a homologous amino acid sequence has no more than 15, nor more than 10, nor more than 5 or no more than 3 conservative amino acid substitutions. In some embodiments, a nucleotide or amino acid sequence has at least 60%, at least 65%, at least 70%, at least 80%, or at least 85% or greater percent identity to a sequence described herein. In some embodiments, a nucleotide or amino acid sequence has at least 90%, 91%, 92%, 93%, 94%, 95%, 96%, 97%, 98% or 99% identity to a sequence described herein. Percent identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). Algorithms suitable for determining percent sequence identity include the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (see the internet at ncbi.nlm.nih.gov). The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4.

In some embodiments, a nucleic acid sequence is codon optimized for expression in a particular species, for example, a mouse sequence can be codon optimized for expression in humans (expression of the protein encoded by the codon-optimized nucleic acid sequence). Thus, in some embodiments, a codon-optimized nucleic acid sequence has at least 60%, at least 65%, at least 70%, at least 80%, or at least 85% or greater percent identity to a nucleic acid sequence described herein. In some embodiments, a codon-optimized nucleic acid sequence acid sequence has at least 90%, 91%, 92%, 93%, 94%, 95%, 96%, 97%, 98% or 99% identity to a sequence described herein.

The term “express” refers to the production of a gene product (e.g., a protein). The term “transient” when referring to expression means a polynucleotide is not incorporated into the genome of the cell. The term “stable” when referring to expression means a polynucleotide is incorporated into the genome of the cell, or a positive selection marker (i.e., an exogenous gene expressed by the cell that confers a benefit under certain growth conditions) is utilized to maintain expression of the transgene.

The term “cytokine” or “cytokines” refers to the general class of biological molecules which affect cells of the immune system. Exemplary cytokines include but are not limited to interferons and interleukins (IL)—in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited or no ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In one embodiment, the vector is a viral vector, e.g. adenovirus. Viral vectors are well known in the art.

As used herein, the term “targeted,” when referring to protein expression, is intended to include, but is not limited to, directing proteins or polypeptides to appropriate destinations in the cell or outside of it. The targeting is typically achieved through signal peptides or targeting peptides, which are a stretch of amino acid residues in a polypeptide chain. These signal peptides can be located anywhere within a polypeptide sequence, but are often located on the N-terminus. Polypeptides can also be engineered to have a signal peptide on the C-terminus. Signal peptides can direct a polypeptide for extracellular section, location to plasma membrane, golgi, endosomes, endoplasmic reticulum, and other cellular compartments. For example, polypeptides with a particular amino acid sequence on their C-terminus (e.g., KDEL) are retained in the ER lumen or transported back the ER lumen.

As used herein, the term “target,” when referring to targeting of a tumor, refers to the ability of NK-92 cells to recognize and kill a tumor cell (i.e., target cell). The term “targeted” in this context refers, for example, to the ability of a CAR expressed by the NK-92 cell to recognize and bind to a cell surface antigen expressed by the tumor.

As used herein, the term “transfect” refers to the insertion of nucleic acid into a cell. Transfection may be performed using any means that allows the nucleic acid to enter the cell. DNA and/or mRNA may be transfected into a cell. Preferably, a transfected cell expresses the gene product (i.e., protein) encoded by the nucleic acid.

The term “suicide gene” refers to a transgene that allows for the negative selection of cells expressing that transgene. A suicide gene is used as a safety system, allowing the cells expressing the gene to be killed by introduction of a selective agent. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene (see also, for example, Yazawa K, Fisher W E, Brunicardi F C: Current progress in suicide gene therapy for cancer. World J. Surg. 2002 July; 26(7):783-9). In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

What is claimed is:

1. A genetically modified NK cell comprising one or more recombinant nucleic acids encoding and expressing
(i) a membrane bound recombinant chimeric antigen receptor (CAR) that comprises in a single polypeptide chain an extracellular binding domain that specifically binds to an EGFR superfamily receptor, a hinge domain, a transmembrane domain, and a FcεRIγ signaling domain having at least 90% sequence identity to the polypeptide sequence of SEQ ID NO:1;
(ii) a suicide gene product that allows the genetically modified NK cell expressing the suicide gene to be killed by contacting the genetically modified NK cell with a selective agent; and
(iii) a recombinant CD16 and/or an autocrine growth stimulating cytokine.

2. The genetically modified NK cell of claim 1, wherein the EGFR superfamily receptor is EGFR.

3. The genetically modified NK cell of claim 2, wherein the extracellular binding domain has at least 90% sequence identity to the polypeptide sequence of SEQ ID NO:41.

4. The genetically modified NK cell of claim 1, wherein the EGFR superfamily receptor is HER2/neu.

5. The genetically modified NK cell of claim 4, wherein the extracellular binding domain has at least 90% sequence identity to the polypeptide sequence of SEQ ID NO:43.

6. The genetically modified NK cell of claim 1, wherein the genetically modified NK cell expresses the recombinant CD16 and the autocrine growth stimulating cytokine, and optionally wherein the recombinant CD16 is a CD16^{158V} mutant.

7. The genetically modified NK cell of claim 1, wherein the autocrine growth stimulating cytokine is IL-2 or IL-15, and optionally wherein the autocrine IL-2 or IL-15 further comprises an endoplasmic retention sequence.

8. The genetically modified NK cell of claim 1, wherein the suicide gene is selected from the group consisting of a thymidine kinase (TK) gene, a cytosine deaminase (CD) gene, a nitroreductase (NR) gene, a xanthine-guanine phosphoribosyl transferase (E. coli gpt) gene, a purine nucleoside phosphorylase (E. coli Deo) gene, a cytochrome P450 gene, or an inducible caspase-9 (iCasp9) gene.

9. The genetically modified NK cell of claim 1, wherein the nucleic acid further encodes and the cell expresses one of IL-12, a TGF-beta trap, or a homing receptor.

10. The genetically modified NK cell of claim 9, wherein the IL-12 is a single chain IL-12 heterodimer.

11. The genetically modified NK cell of claim 9, wherein the TGF-beta trap comprises a single chain dimer of the TGF-beta Receptor II ectodomain, or wherein the TGF-beta trap is a secreted form of a single chain dimer of the TGF-beta Receptor II ectodomain.

12. The genetically modified NK cell of claim 9, wherein the homing receptor is selected from the group consisting of CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCXCKR, D6, DARC, and a receptor for CXCL14.

13. The genetically modified NK cell of claim 1, wherein the FcεRIγ signaling domain has the polypeptide sequence of SEQ ID NO:1.

14. The genetically modified NK cell of claim 1, wherein the extracellular binding domain has at least 90% sequence identity to the polypeptide sequence of SEQ ID NO:41, or wherein the extracellular binding domain has at least 90% sequence identity to the polypeptide sequence of SEQ ID NO:43.

15. The genetically modified NK cell of claim 1, wherein the extracellular binding domain has the polypeptide sequence of SEQ ID NO:41, or wherein the extracellular binding domain has the polypeptide sequence of SEQ ID NO:43.

16. The genetically modified NK cell of claim 15, wherein the suicide gene is selected from the group consisting of a thymidine kinase (TK) gene, a cytosine deaminase (CD) gene, a nitroreductase (NR) gene, a xanthine-guanine phosphoribosyl transferase (E. coli gpt) gene, a purine nucleoside phosphorylase (E. coli Deo) gene, a cytochrome P450 gene, or an inducible caspase-9 (iCasp9) gene.

17. A method of treating cancer in a patient in need thereof, comprising administering to the patient a therapeutically effective amount of the genetically modified NK cells of claim 1 and thereby treating the cancer, wherein the genetically modified NK cell is autologous or allogeneic to the patient.

18. The method of claim 17 further comprising a step of administering at least one additional therapeutic entity selected from the group consisting of a viral cancer vaccine, a bacterial cancer vaccine, a yeast cancer vaccine, N-803, an antibody, a stem cell transplant, and a tumor targeted cytokine.

19. The method of claim 17, wherein the cancer is a lung cancer, a breast cancer, a thyroid cancer, an esophageal cancer, a gastric cancer, a gastroesophageal cancer, or a head and neck cancer.

20. The method of claim 17, wherein about 1×10⁸ to about 1×10¹¹ cells per m² of body surface area of the patient are administered to the patient.
